(19) United States
(12) Patent Application Publication
(10) Pub. No.: US 2025/0129319 A1
(43) Pub. Date: Apr. 24, 2025

(54) POLYMER MEMBRANE AND METHOD OF MANUFACTURING THE SAME

(71) Applicant: KONINKLIJKE PHILIPS N.V., EINDHOVEN (NL)

(72) Inventors: Lucas Johannes Anna Maria Beckers, Veldhoven (NL); Renatus Hendricus Maria Sanders, Roermond (NL)

(21) Appl. No.: 18/689,172

(22) PCT Filed: Sep. 21, 2022
(86) PCT No.: PCT/EP2022/076211

§ 371 Date: Mar. 5, 2024

(30) Foreign Application Priority Data
Sep. 21, 2021 (EP) 21198126.1

(51) Int. Cl. C12M 1/12

(52) U.S. Cl. CPC C12M 25/04

(57) ABSTRACT

Provided is a method (100) of fabricating a polymer membrane (206) suitable for a cell culture device. The method comprises providing (102) a first polymer (200) having a first surface (201), and providing (104) a solution (204) on the first surface. The solution comprises a second polymer, a surfactant, and a solvent. The surfactant comprises surfactant molecules each having a polar moiety. The method further comprises evaporating (106) the solvent while the solution is on the first surface to provide the polymer membrane. The polymer membrane has a second surface (207) in contact with the first surface. At least the first surface of the first polymer has greater affinity for the polar moiety than the second polymer. Further provided is the polymer membrane per se, a fluidic device, such as a cell culture device, comprising such a polymer membrane, and a cell culturing method comprising encapsulating cells with one or more of the polymer membrane.

### FIELD OF THE INVENTION

This disclosure relates to a method of fabricating a polymer membrane, in particular a polymer membrane suitable for a cell culture device.

The disclosure also relates to a polymer membrane for a cell culture device.

The disclosure further relates to a cell culture device, such as for example a fluidic device, comprising such a polymer membrane.

The disclosure yet further provides a cell culturing method comprising encapsulating cells with one or more of such a polymer membrane.

### BACKGROUND OF THE INVENTION

In vitro testing of mammalian cells or tissue is an important technique to obtain clinically important information of the mammalian material under investigation. For example, biopsied mammalian cell or tissue material may be subjected to such testing in order to determine anomalies or disease in such mammalian material or to expose diseased mammalian material to drugs, e.g. experimental drugs, to monitor the response of the diseased mammalian material to such exposure. This approach, for example, is frequently used in oncological procedures. This can provide important insights in how a disease in an individual can be effectively treated without having to expose the individual to a range of potentially effective drugs, which can be undesirable for a number of reasons, such as drug toxicity. In addition, the efficacy of experimental drugs for existing diseases for which no established satisfactory drug treatment is yet available may be tested in this manner. There are many other well-known reasons for deploying such in vitro tests.

A common approach to such in vitro testing is to immobilize the mammalian material in a fluidic device, which is sometimes referred to as an organ-on-chip. In such an approach, the mammalian material is typically immobilized on a membrane separating two fluidic channels of the fluidic device, with a first channel being used to feed the mammalian material and the second channel being used to expose the mammalian material to a chemical compound or composition of interest such as a drug treatment, e.g. to test the efficacy and/or toxicity of the drug as previously explained. The fluidic device, or at least its membrane, may be made of an elastomer such that the fluidic device may be diced or sliced in order to obtain a slice of the membrane including the mammalian material for evaluation purposes, e.g. to evaluate the reaction of the mammalian material to the exposure to the chemical compound or composition of interest.

Cell samples, such as spheroids/organoids and biopsies, are often studied in well cell plates. Fluids are injected and taken away with pipets under visual inspection. The spheroids/organoids and biopsies are intended to stay in the wells. The use of fluidic devices where biopsies are placed on membranes is the latest state of the art practice. Fluidic handling protocols are currently in development. Fluids, e.g. containing cell nutrients and/or experimental drug(s), are passed over such membranes, and can risk sweeping spheroids/organoids away.

Another challenge in such in vitro testing is to ensure that the mammalian material, e.g. cells or tissue, are stabilized and develop normally on the membrane of the fluidic device such that they live for the duration of the test procedure. This is particularly challenging in oncology procedures where experiments can be rather lengthy. To this end, the mammalian material may be stabilized using a biocompatible material such as fibronectin, which ensures that the mammalian cells will keep multiplying by providing the cells with a chemical environment mimicking that of natural tissue.

Soft rubber or polymeric materials for the membrane are relatively apolar and hydrophobic, and do not generally have functional groups to adhere to or bond proteins. The implementation of soft rubber substrates or membranes in fluidic devices for cell culturing has generally been found to be difficult and expensive. Coatings or extra cellular matrices have also been found to be too easily flushed away in fluidic devices.

### SUMMARY OF THE INVENTION

The invention is defined by the claims.

According to examples in accordance with an aspect of the disclosure herein there is provided a method of fabricating a polymer membrane suitable for a cell culture device. The method comprises: providing on a first surface of a first polymer: a surfactant comprising surfactant molecules each having a polar moiety; and a second polymer. The method further comprises forming the polymer membrane to comprise at least part of the surfactant molecules and the second polymer, the polymer membrane having a second surface (207) in contact with the first surface: the first polymer, the second polymer and the surfactant molecules being chosen such that the polar moiety has a greater affinity for the first surface than for the second polymer.

The surfactant molecules include a polar moiety typically connected to a less polar or non-polar moiety, i.e. the polar moiety is more polar than the less-polar or non-polar moiety. The polar moiety may be a hydrophilic moiety and the non-polar moiety a hydrophobic moiety, which means that the hydrophilic moiety is more hydrophilic than the hydrophobic moiety. Such surfactant molecules may thus comprise or consist of amphiphilic molecules.

By at least the first surface, and in some examples the entirety, of the first polymer having greater affinity for the polar moiety than the second polymer, a preponderance of the surfactant molecules at the second surface of the polymer membrane may be orientated such that the polar moiety is made available at the second surface. The polar moieties cause the surfactant molecules to be capable of assembly at the first surface before the formation of the polymer membrane.

A higher affinity of the first polymer, or at least the first surface of the first polymer, relative to the second polymer for the polar moiety can be achieved by selecting a first polymer whose polarity and/or hydrophilicity, at least at the first surface, is higher than that of the second polymer. Alternatively or additionally, the first polymer may be selected to form stronger intermolecular interactions, such as hydrogen bonds, with the polar moiety at the first surface than the second polymer. The adhesion of the parts other than the polar moiety of the surfactant molecules thus preferably have a higher affinity for the second polymer than for the first polymer.

By the polar moiety of the surfactant molecules being made available at the second surface of the polymer membrane in this manner, the surface properties of the second surface may be particularly suitable for binding of, for example, a cell culturing protein, such as fibronectin, thereto. Additionally or alternatively, the second surface may be directly suitable for cell culturing without providing it with a culturing protein. The usefulness of such functionalised or non-functionalised surfaces for ceel culturing have been described in for example WO 2019015988. WO20200148427 and WO2021058657, which are both incorporated by reference in their entirety herein.

Moreover, due to the first polymer. e.g. in the form of a foil or other body, being employed for effecting the above-described orientation of the polar moiety, the polymer membrane can be relatively straightforwardly released, or removed, or separated therefrom, for instance by peeling the first polymer and the polymer membrane from each other, and/or by dissolving the first polymer in a solvent in which the polymer membrane is less soluble or insoluble so as to separate the polymer membrane from the first polymer. This provides a more convenient method of fabricating a polymer membrane having polar moieties available at a surface thereof.

Furthermore, the method makes it possible to efficiently manufacture fragile and relatively thin polymer membranes (e.g. with a thickness of less than 100 micrometer) without them disintegrating. Especially, the dissolution based removal of the first polymer is beneficial in this respect as it avoids the need for relatively high pulling forces exerted during peeling.

During the forming of the polymer membrane, the surfactant molecules are made to adhere to the polymer membrane more strongly than to the first surface by any of their moieties different from the polar moiety. Hence during any release or separation of the first polymer from the polymer membrane at least some surfactant molecules remain adhered to the polymer membrane at the second surface to provide the desired and herein described effects. Adherence in this respect can occur via non-covalent and/or covalent bonding, of which the latter is preferred, as will be further described herein. The first and second polymer are chosen such that after the polymer membrane has been formed the second polymer and/or polymer membrane does not covalently bond to the first polymer.

The surfactant molecules may be provided to the first surface before the second polymer is provided. e.g. in a sequence of application steps. Alternatively, they may be provided at the same time. e.g. as a mixture in a composition. Any methods suitable may be used for this. For example, solvents and/or dispersants may be used to provide them to the first surface. To this end, the surfactant may comprise a solvent or dispersant (each of which can be a pure solvent or a mixture of several solvents) for dissolving or dispersing the surfactant molecules in. The surfactant may be spincoated or spraycoated or otherwise provided to the first surface. The solvent or dispersant preferably is evaporated or otherwise removed from the first surface after having provided the surfactant thereto. The second polymer may be provided to the first surface using a solvent or dispersant for dissolving or dispersing the second polymer. The solvent or dispersant may be the same as, or different from that of the surfactant. If surfactant molecules and second polymer are to be successively provided to the first surface, the second polymer solvent may be advantageously chosen to not substantially dissolve or disperse the previously provided surfactant molecules so that the surfactant molecules remain substantially located/assembled near the second surface in stead of becoming distributed over the bulk and away from the second surface of the polymer membrane. However, if such effect is not needed or bulk distribution is desired, then such solvent differentiation is not needed. The solvent and or dispersant for the surfactant and the second polymer can be the same. The method may then comprise providing a composition comprising the surfactant, the second polymer, and the solvent or dispersant. The latter may be advantageous when it is desired to have some of the surfactant molecules distributed over the bulk of the polymer membrane instead of predominantly or only assembled at its second surface. Furthermore, it reduces the number of steps and increases manufacture efficiency.

The forming of the polymer membrane may comprise evaporating the solvent or dispersant for the second polymer, and/or may comprise evaporating the solvent or dispersant for the surfactant molecules. In one example the method comprises the providing a solution on a first surface of a first polymer, the solution comprising the second polymer, the surfactant, and the solvent, wherein the surfactant comprises surfactant molecules each having a polar moiety; and evaporating the solvent while the solution or dispersion resides on the first surface to provide said polymer membrane.

The surfactant may comprise multiple types of the surfactant molecules in that surfactant molecules may have different type of polar moieties and/or different type of non-polar moieties.

In some embodiments, the second polymer comprises an elastomer. Alternatively or additionally, the second polymer has a Shore A hardness of 70 or less, more preferably 60 or less, and most preferably 20 or less. The deformation. e.g. resilient deformation, of such a second polymer can be particularly advantageous when the polymer membrane is employed in a cell culture device because the second polymer can be deformed. e.g. resiliently deformed, against a substrate and/or cover plate in order to seal fluid within the cell culture device.

The polymer membrane may comprise a frame for at least partly supporting the polymer membrane at some of its edges. Such frame may be made of metal or plastic. The frame may facilite handling of the membrane without breaking it and/or mounting of the membrane in a cell culturing device.

Preferably, the second polymer comprises or consists of a polydiene, such as polybutadiene, polybutadienestyrene, polyisoprene or butyl rubber. Particular mention is made of polybutadiene due to its chemical and mechanical properties making it particularly suitable for employment in a cell culture device. Alternatively, or additionally, the second polymer comprises or consists of a polysiloxane. These types of second polymers typically may provide the elastomer. They typically may also be transparent which is useful for cell culturing membranes or devices.

Moreover, in non-limiting examples in which the surfactant molecules, e.g. the hydrophobic moiety thereof, comprise an unsaturated carbon-carbon bond, the unsaturated carbon-carbon bonds included in the polybutadiene can provide sites at which the surfactant molecules can be grafted, for example covalently bonded, to the polybutadiene second polymer.

More generally, the method may further comprise crosslinking the second polymer and at least some of the surfactant molecules. The surfactant molecules preferably have one or more reactive groups different from the polar moiety to engage in such crosslinking. Preferably such one or more reactive groups are chosen such that they do not react with the first polymer. In addition the second polymer comprise one or more further reactive groups for reacting with the one or more reactive groups to form the crosslinks. Preferably such groups form covalent bonds to form such crosslinks.

The second polymer can comprise an additive for assisting the crosslinking, such as for example a catalyst for catalysing the reaction between the reactive and further reactive groups. Examples are described herein below.

The second polymer may for example comprise one or more unsaturated carbon-carbon bonds. Preferably such bond is chosen from the group consisting of alkenyl and alkynyl bonds. More preferably such bond is chosen from the group consisting of alkenyl bonds. Preferably the carbon-carbon double bonds are non-conjugated with other double bonds. Preferably the one more double bonds comprise at least one terminal such bond.

The second polymer may, for example, comprise an unsaturated carbon-carbon bond. e.g. a vinyl functional group, for example as in the case of polybutadiene, and/or a hydride functional group, and the at least some of the surfactant molecules each comprise an unsaturated carbon-carbon bond in the hydrophobic moiety, e.g. as in the case of the above-mentioned unsaturated surfactant, such as the unsaturated fatty acid. In such a non-limiting example, the crosslinking may be via a reaction between the unsaturated carbon-carbon bond of the surfactant and the unsaturated carbon-carbon bond, e.g. vinyl functional group, and/or hydride functional group of the second polymer.

More generally, the second polymer may comprise or consist of at least one selected from the group consisting of: a polydiene, a polysiloxane, such as polydimethylsiloxane (PDMS), and, optionally, a polyurethane.

A polysiloxane can, similarly to the above-described polydiene, have unsaturated carbon-carbon bonds (and/or hydride functional groups). This may enable the polysiloxane to crosslink with surfactant molecules comprising an unsaturated carbon-carbon bond.

It is noted that the lack of unsaturated carbon-carbon bonds in the case of, for example, a polyurethane second polymer may mean that this particular second polymer may not form crosslinks with surfactant molecules comprising an unsaturated carbon-carbon bond.

In some embodiments, the first polymer comprises or consists of one or more of a polyol such as e.g. polyvinylalcohol, polyurethane and polyethylene glycol. Such first polymers, e.g. in the form of a foil, can be releasable from the polymer membrane. For example using the peeling or dissolution methods. The polyols typically are soluble in polar solvents such as water or alcohol, whereas the second polymer and the polymer membrane are not.

The method may comprise release of the polymer membrane form the first surface. Such release can be implemented in any suitable manner, noting, in particular, the properties of the first polymer as described herein. For example, such release may be implemented by peeling and/or dissolving of the first polymer. When peeling is used for the release, the first polymer and second polymer are chosen such that they do not have a high affinity towards each other. For example, the second polymer is chosen to be less polar (e.g. less hydrophilic) than the first polymer. When dissolving is used for the release, the release solvent is chosen such that it is capable of dissolving the first polymer, but not substantially the polymer membrane. In other words at least the second polymer and the surfactant molecules at the second surface are not dissolved by the release solvent. Preferably, taking account of conditions during release, it is advantageous to have the surfactant molecules covalently bonded to the second polymer or polymer membrane.

It is noted that such dissolving can be used, for instance, in the case of polyvinyl alcohol and polyethylene glycol as the first polyer, whereas the relatively low solubility of polyurethane may mean that peeling or stripping of a polyurethane first polymer from the polymer membrane is used instead of dissolving.

Moreover, such a polyvinylalcohol, polyurethane or polyethylene glycol first polymer may be more polar and/or form stronger intermolecular interactions, such as hydrogen bonds with, and thus have greater affinity for, the polar moiety than the second polymer, for example when the second polymer is a polydiene, such as polybutadiene.

In at least some embodiments, the solvent comprises or consists of one or more of a hydrocarbon solvent, an aliphatic hydrocarbon solvent, such as an alkane solvent such as e.g. heptane, an alcohol solvent, such as isopropyl alcohol, and a ketone solvent, such as acetone. For example, the solvent comprises or consists of heptane, for example when the second polymer comprises or consists of a polydiene, such as polybutadiene.

The polar moiety may be a Bronsted-Lowry acid/base. These include acids capable of exchanging protons with appropriate conjugated bases as known in the art.

The polar moiety may be a carboxylic acid group, a phosphoric acid group, a phosphorous acid group, a sulphonic acid group, an amine group or an amide group. Preferably, the polar moiety may be a carboxylic acid group, a phosphoric acid group, a phosphorous acid group, an amine group or an amide group. More preferably, the polar moiety is a carboxylic acid group, an amine group or an amide group. Such functional groups can interact with, e.g. covalently bond to, a cell culturing protein. Also, these groups may allow cell culturing without covalently bonding of a cell culturing protein. For example they may cause adherence of cell culturing proteins to them that are present and/or formed in cell culturing media during the cell culturing. Refer for example to WO 2019015988, WO20200148427 and WO2021058657.

The polar moieties may be one or more acidic groups chosen from the group consisting of: carboxylic acid groups, phosphoric acid groups and phosphorous acid groups.

The non-polar moieties may comprise one or more groups chosen from the group consisting of: linear and branched hydrocarbon chains, such as for example aliphatic chains, and linear and branched polyether chains.

The non-polar moieties preferably comprise one or more reactive groups capable of forming a covalent bond with the second polymer. Such reactive group is thus less polar than the polar moiety. The one or more reactive groups preferably are chosen from the group consisting of: imide, carbonyl, alkenyl, such as for example vinyl, alkynyl, all of which may be present as terminal and/or non terminal groups, where the latter is sometimes preferred with respect to increased reactivity as compared to non-terminal groups. Preferably the groups are non-conjugated.

In some embodiments, the surfactant comprises a fatty acid, with the carboxylic acid group of the fatty acid defining the polar moiety.

In embodiments in which the surfactant comprises a fatty acid, the fatty acid may comprise or consist of an unsaturated fatty acid. The unsaturated carbon-carbon bond(s) of such a fatty acid may, for example, be used to crosslink the unsaturated fatty acid to the second polymer, e.g. via the above-described reaction involving the unsaturated carbon-carbon bond and/or hydride functional group included in the second polymer.

The unsaturated fatty acid may be one or more selected from myristoleic acid, palmitoleic acid, sapienic acid, oleic acid, elaidic acid, vaccenic acid, linoleic acid, linoelaidic acid, α-linolenic acid, arachidonic acid, eicospaentaenoic acid, erucic acid and docosahexaenoic acid. Particular mention is made of linoleic acid, e.g. α-linolenic acid. As a vinyl group containing non-saturated fatty acid Undec-10-enoic acid may serve as a preferred example.

The method may comprise separating the polymer membrane and the first polymer. Such separating can be implemented in any suitable manner. For example, the separating can comprise delaminating, e.g. peeling, the first polymer from the polymer membrane and/or by dissolving the first polymer in a solvent in which the polymer membrane is less soluble or insoluble so as to separate the polymer membrane from the first polymer. The method using the dissolution is advantages for fragile polymer membranes such as thin membranes.

In at least some embodiments, the method comprises forming one or more apertures and/or grooves in the polymer membrane. Such groove(s) and/or aperture(s) may assist the polymer membrane to fulfil a function in, for instance, a cell culture device, such as enabling nutrients to be supplied and waste products to be removed from the cells via the groove(s) and/or aperture(s), and/or enabling monitoring of a cell sample.

The size/diameter of the apertures 210 can be selected according to what is most practical, e.g. for handling and/or microscopy. Such aperture(s) may, for example, have a diameter in the range of 1-1000 μm, preferably in a range of 1-100 μm, more preferably in a range of 1 to 20 μm or even 1-10 μm.

The forming of apertures and/or grooves may, comprise laser drilling the polymer membrane. This may be done before removal of the first polymer or after. Thus, any of the methods disclosed herein can comprise forming one or more apertures and/or grooves in the polymer membrane, using laser ablation with a femto second or picosecond laser. Preferably, in such case, the apertures and/or grooves comprise at least one opening dimension measured in the plane of the polymer membrane in a range of less than 50, preferably less than 20 micrometer. In some examples the wavelength of the femtosecond or picosecond laser is between 400 and 1200 nm. Preferably the polymer membrane laser drilled comprises a polymer chosen from the group consisting of a polybutadiene and a polysiloxane, the polymer membrane having a thickness of less than 100 micrometer.

It is noted that the method of laser drilling for forming the apertures or grooves as described herein can be used independent of the method of making the polymer membrane as described herein. Thus, otherwise made polymer membranes, preferably with a thickness of less than 100 micrometer and preferably comprising a polysiloxane and/or polybutadiene may be laser drilled as described herein.

In other embodiments the forming of apertures and/or grooves may comprise using the relief of the first polymer at the first surface as a mold for forming a relief within the polymer membrane at the second surface. Thus, the first surface can have a relief such that it comprises protrusions defining for example the apertures and/or indentations in the polymer membrane once the polymer membrane is released from the first surface. The relief comprising first surface can have a protrusion for defining an indentation (not a hole) in the second surface of the first polymer. Hence such first surface can be used to prepare the second surface as a relief surface with indentations in stead of or in addition to holes in the polymer membrane. Such indentations can provide specific functions during cell culturing. One example would be indentations to grow organoids in. The first surface may be chosen to have a relief surface having one or more protrusion for defining indentations with one or more apertures in the polymer membrane at the second surface.

In some embodiments, the method comprises functionalizing the second surface of the polymer membrane with a cell culturing protein, e.g. a human protein. The above-described polar moieties made available at the second surface facilitate this functionalization. For example through polar interactions or covalent bonding to the protein.

The cell culturing protein can, for example, comprise or consist of one or more of collagen, elastin, and fibronectin. In the case of fibronectin, this protein can be straightforwardly coupled to the polar moiety, particular in examples in which the polar moiety is a carboxylic acid group. In some embodiments, the method includes providing a cell culturing device body having one or more cavities and mounting one or more polymer membranes in at least some of the one or more cavities. Preferably such mounting is such that each of the at least some of the one or more cavities are separated by the polymer membrane in a first cavity and a second cavity. Preferably the cell culturing device body comprises first and or second fluidic channels connected to the first and/or second cavities. Preferably the polymer membranes are supported by frames at their outer circumference. The body may be a well cell plate (e.g. a multi-well plate) for example made of polystyrene. The body may be an elastomeric body for example made of PDMS. The method may comprise clamping the elastomeric body between cover plates such as e.g. made of glass. Examples of cell culturing devices in the form of e.g. fluidic devices have been disclosed in WO2019015988 and WO2021058657, which are incorporated by reference in their entirety herein.

According to another aspect there is provided a polymer membrane obtainable from the method defined above.

According to still another aspect there is provided a cell culturing device comprising, or in the form of a fluidic device, the device comprising one or more polymer membranes according to the present disclosure. A fluidic device being understood to comprise at least one channel for allowing or providing a fluid flow towards and/or away from the polymer membrane. In some embodiments the fluidic device comprises a first cavity separated from a second cavity by the polymer membrane. Preferably there is a first flow channel connected to the first cavity and a second flow channel connected to the second cavity, each flow channels having one or more openings to provide external fluid flows to the fluidic device. There may be a plurality of ensembles of such membrane separated cavities. Multiple first cavities may be connected using the same first fluid channel and/or multiple of the second cavities may be connected using the same second fluid channels.

Exemplifying such cell culturing devices or fluidic device are based on designs as have been disclosed in WO2019015988 and WO2021058657 where the membranes are now replaced with those of the current disclosure by e.g. mounting such membrane in a cavity of the designs disclosed.

According to yet another aspect there is provided a cell culturing device, comprising: a polymer membrane and a further polymer membrane, the polymer membrane having a surface which opposes a further surface of the further polymer membrane, at least one of the polymer membranes comprising a surfactant comprising surfactant molecules each having a polar moiety at the respective surface or surfaces. The cell culturing device may comprise or consist of a fluidic device.

Encapsulating cells between the polymer membranes in this manner may lessen the risk of such cells. e.g. of a biopsy or spheroid/organoid, being flushed away when a fluid is provided to and/or withdrawn from the fluidic device.

One or both of the polymer membranes may, for example, be optically transparent, such as to enable optical analysis of the interior of the fluidic device between the polymer membranes. Such optical analysis can, for instance, be carried out using a suitable microscope.

A cell culturing protein may be bound to said one or both of the surfaces via the polar moiety, e.g. via covalent bonding of the cell culturing protein to the polar moiety.

One, or preferably both, of the polymer membranes may comprise one or more grooves and/or apertures. Such aperture(s) may, for example, have a diameter in the range of 1-1000 μm. Preferably, in the range of 1-100 μm, or 1-50 μm. More preferably in the range of 1-20 μm.

Aperture(s) in one or both of the membranes can, for instance, be optimized for a cell sample or type of cell sample, e.g. according to the dimensions of cells and/or cell layer(s) and/or spheroid(s)/organoid(s) and/or biopsy/ies.

In a non-limiting example, both polymer membranes are made of the same polymer, e.g. a polydiene, such as polybutadiene.

According to a further aspect there is provided a cell culturing method comprising immobilizing cells within a cell culturing device using at least one of the polymer membrane according to the present disclosure. The immobilizing may comprise fixating and/or partially or entirely encapsulating the cells using the polymer membrane.

More generally, embodiments described herein in relation to the polymer membrane, fluidic device and cell culturing method may be applicable to the method of manufacturing the polymer membrane, and embodiments described herein in relation to the method of manufacturing the polymer membrane may be applicable to the polymer membrane itself, as well as the fluidic device and cell culturing method.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIGS. 1A and 1B schematically depict a method of manufacturing a polymer membrane according to an example:

FIG. 2A schematically depicts orientating of surfactant molecules such as to make a polar group thereof available at a surface of the polymer membrane:

FIG. 2B schematically depicts polysiloxane curing and crosslinking with an unsaturated fatty acid surfactant taking place on a polymer surface:

FIG. 3 provides a plan view of an exemplary polymer membrane and a frame:

FIGS. 4A and 4B provide micrographs of part of an exemplary polymer membrane:

FIG. 5 provides a micrograph of another exemplary polymer membrane:

FIGS. 6A and 6B schematically depict encapsulation of a tissue sample by a plurality of polymer membranes:

FIG. 7 schematically depicts encapsulation of spheroids/organoids by a plurality of polymer membranes:

FIGS. 8A to 8C provide micrographs of an exemplary polymer membrane for cell sample encapsulation; and

FIG. 9 provides a micrograph of an exemplary linoleic acid-modified polysiloxane polymer membrane.

FIG. 10 provides a method of mounting a polymer membrane in fluidic module to make a fluidic device.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

Provided is a method of fabricating a polymer membrane suitable for a cell culture device.

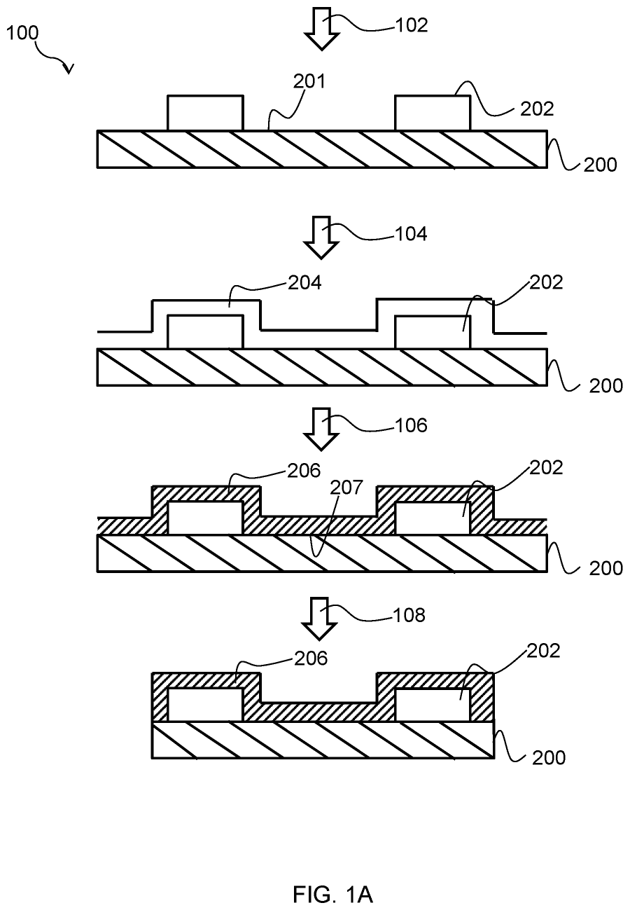

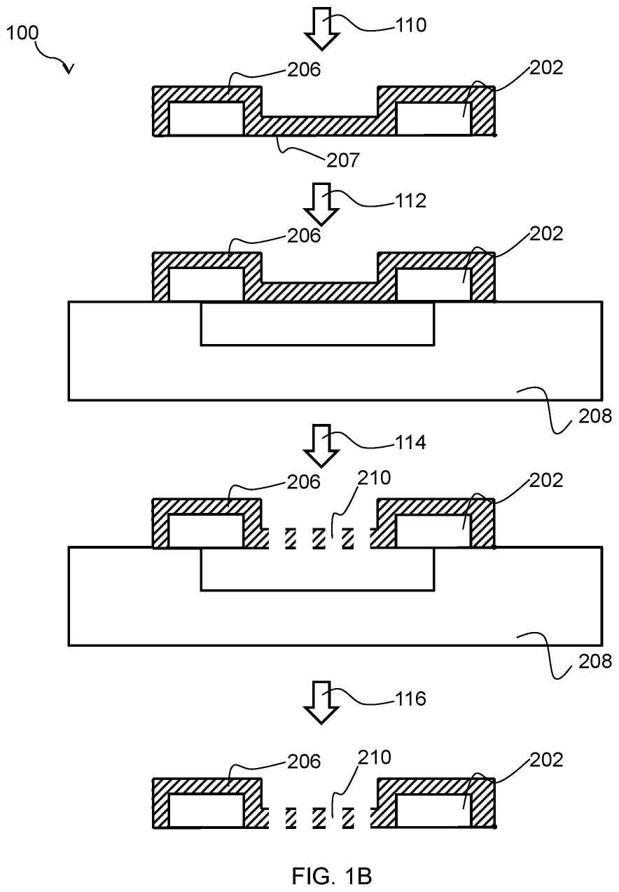

FIGS. 1A and 1B schematically depict a method 100 for fabricating a polymer membrane according to a non-limiting example. The method comprises providing 102 a first polymer 200 having a first surface 201. In this case the first surface is flat, but this is not necessary. Depending on a desired relief structure of the polymer membrane with or without holes therein, the first surface may be shaped to serve as a mold for such desired relief. It may thus have the appropriate indentations, protrusions as features of the relief with desired width, length height or depth.

A frame 202 is also provided on the first polymer 200, in particular on the first surface 201. The frame 202 can facilitate subsequent manufacturing steps, such as forming one or more grooves and/or apertures in the polymer membrane, as will be described in more detail herein below. Also, the frame may facilitate handling of the membrane once fabricated and attached to it In other examples, such a frame 202 can be introduced at a different stage of the method 100, or omitted altogether.

The method 100 comprises providing 104 a solution or dispersion 204 on the first surface 201 and optionally, but preferably also on the frame 202. The solution comprises a second polymer, a surfactant, and a solvent. The second polymer corresponds at least partly to the polymeric component of the polymer membrane. In this case a solvent is used in the step of providing the components to the first surface. This is however not necessary. Also the components are provided to the first surface at the same time. This is however also not necessary and for example the surfactant may be provided to the surface before the second polymer is provided.

The providing 104 the solution 204 can be implemented in any suitable manner, such as by dispensing, spin coating, etc. The manner in which the solution 204 is provided 104 can, for example, determine the thickness of the polymer membrane, as will be discussed in more detail herein below.

The surfactant comprises surfactant molecules each having a polar moiety, which may be a hydrophylic moiety. The surfactant can also include a hydrophobic moiety which is less polar than the polar moiety.

The polarities of the polar moiety and less polar moieties, and/or hydrophilicity of the hydrophilic and hydrophobic moieties are such as to cause the surfactant molecules to at least partly assemble at the first surface once applied to it, even during application of the second polymer to the first surface. Without wanting to be bound by theory, this assembly may be at least partly due to the first surface being polar (e.g. hydrophylic) capable of attracting the polar moieties of the surfactant molecules. The assembly may involve a partial orientation of the surfactant molecules close to the first polymer surface with their polar moieties facing the polar first surface and their less polar hydrophobic moieties directed away from the polar surface.

The polar moiety, once the polymer membrane has been formed, may also provide a site to which, for example, a cell culturing protein, such as fibronectin, can bind, for example by grafting. e.g. covalent bonding, to the polymer membrane. Such binding of the cell culturing protein to the polymer membrane can, in at least some examples, be implemented in a later step of the method 100.

In some embodiments, the polar moiety comprises one or more moieties chosen form the group consisting of: a carboxylic acid groups, phosphoric acid groups, amine groups and amide groups. Such moieties or functional groups can interact with, e.g. covalently bond to, a cell culturing protein. However, other moieties are possible as also described herein.

The surfactant molecules hydrophobic section preferably comprises a hydrocarbon chain such as e.g. an aliphatic chain (preferably linear, but may be branched) carrying the polar moiety at some location. The hydrophobic section may comprise or consist of a polyether chain. Preferably the polar moiety is carried at a first terminal end of the aliphatic chain. The aliphatic chain preferably has at least 3 carbon atoms (>C3), more preferably at least 5 carbon atoms (>C5), more preferably at least 7 carbon atoms (>C7). The aliphatic chain preferably has less than 30 carbon atoms (<C30), or less than 25, or less than 20 or even less than 15 carbon atoms (<15C). The hydrocarbon chain may comprise cyclic constituents such as cyclohexadiene units and/or aromatic constituents.

Examples of an amine polar moiety-comprising surfactant are octadecanamide, also known as N-(3-dimethylaminopropyl)octadecamide, and polylysine, e.g. poly-D-lysine.

Preferably, the surfactant comprises, or is in the form of, a fatty acid, with the carboxylic acid group of the fatty acid defining the polar moiety. Preferably it is an unsaturated fatty acid.

More generally, in some embodiments when the surfactant molecules (i.e. the hydrophobic moieties thereof) of any kind described herein comprise a reactive group for covalent bonding to the second polymer and the method 100 further comprises curing the second polymer and/or crosslinking the second polymer to at least some of the surfactant molecules using the reactive group, the polar moieties do not participate in the curing and/or crosslinking reaction. This causes the surfactant molecules to be immobilised to the second polymer at least at the second surface of the polymer membrane. Hence they may e.g. better withstand experimental conditions during cell culturing. i.e. for example may part from the second surface more difficult or not at all due to covalent bonding to therewith cause less or no disturbing effects in any cell culturing process. Crosslinking in this context may refer to covalently binding.

In surfactant molecules as described herein before that can be covalently bonded to the second polymer the aliphatic chain preferably has one or more reactive groups capable of reacting with the second polymer. Such groups may comprise or consist of unsaturated carbon-carbon bonds such as acetylene or vinyl bonds. Preferably these are non-conjugated unsaturated bonds. Preferably, at least one of the one or more unsaturated bonds is at a second terminal end (different form the first terminal end) of the aliphatic chain. Preferably, the unsaturated bond is separated from the polar moiety by at least 3 carbon atoms, more preferably by at least 5 carbon atoms, more preferably by at last 7 carbon atoms.

Such curing can be implemented in any suitable manner. Curing may be implemented with UV radiation and/or thermally, in other words by heating. Curing/crosslinking the second polymer will be described in more detail herein below. Reactions for curing may proceed through cationic, anionic other polar, or radical mechanisms.

In some embodiments, the surfactant molecules. e.g. the hydrophobic moiety thereof, comprise an unsaturated carbon-carbon bond as the reactive moiety. Such an unsaturated carbon-carbon bond can, in some non-limiting examples, be crosslinked to the second polymer.

The above-mentioned fatty acid can, for instance, comprise or consist of an unsaturated fatty acid. The unsaturated carbon-carbon bond(s) in the hydrophobic tail of such an unsaturated fatty acid is or are crosslinkable to the second polymer. Such fatty acid may thus have alkene such as vinyl groups.

Such an unsaturated fatty acid is, for instance, one or more selected from myristoleic acid, palmitoleic acid, sapienic acid, oleic acid, elaidic acid, vaccenic acid, linoleic acid, linoelaidic acid, linoleic acid, e.g. α-linolenic acid, arachidonic acid, eicospaentaenoic acid, erucic acid and docosahexaenoic acid. Undec-10-enoic acid is one example of a fatty acid having a vinylic group.

In the context of the present disclosure, where reference is made to an unsaturated fatty acid, it is to be understood that this is intended to refer to any organic compound comprising a carboxylic acid head group covalently bound to an aliphatic tail comprising 10-30 carbon atoms and at least one carbon-carbon double bond in the aliphatic tail. The aliphatic tail may terminate in a non-aromatic ring structure, e.g. a five-membered or 6-membered cyclopentyl or cyclohexyl group, which may itself contain at least one double bond. A non-limiting example of an organic compound intended to fall within the above definition of an unsaturated fatty acid is retinoic acid. Many other compounds in addition to the example compounds explicitly mentioned in the present disclosure will be immediately apparent to the skilled person.

The second polymer and the surfactant, e.g. fatty acid, such as an unsaturated fatty acid, may be in any suitable ratio. Preferably, the surfactant is present in the solution 204 and/or in the polymer membrane in a range of 0.05-35% by weight of the second polymer. This range may assist the polymer membrane 206 to retain sufficient polymeric character. e.g. deformability.

The second polymer can be any suitable polymer suitable for use in a cell culture device. The second polymer can itself be a biocompatible polymer. Alternatively or additionally the surfactant can itself be biocompatible. In such cases functionalisation of the second surface with biocompatible culturing proteins is not needed perse.

It is nonetheless noted that the second polymer and the surfactant in some cases need not themselves be biocompatible. After all, if subsequent functionalization of the polymer membrane with a cell culturing protein, such as fibronectin occurs, cells are not directly exposed to the second polymer.

In case the surfactant molecules are intended to be cured and/or crosslinked to the second polymer, the latter is chosen to have polymer reactive groups that are capable of reaction with the reactive group of the surfactant molecule to form a covalent bonding between the surfactant molecule and the second polymer. The second polymer typically will then comprise a plurality of such polymer reactive groups for bonding a plurality of surfactant molecules.

The second polymer preferably comprises or consist of an elastomer. Alternatively or additionally, the second polymer has a Shore A hardness of 70 or less, more preferably 60 or less, and most preferably 20 or less. The deformation. e.g. resilient deformation, of such a second polymer can be particularly advantageous when the polymer membrane is employed in a cell culture device because the second polymer can be deformed. e.g. resiliently deformed, against a substrate (not visible in FIGS. 1A and 1B) and/or cover plate in order to seal fluid within the cell culture device.

In some embodiments, the second polymer comprises or consists of at least one selected from a polydiene, such as polybutadienestyrene, a polysiloxane, such as polydimethylsiloxane (PDMS), and, optionally a polyurethane.

Preferably, the second polymer comprises or consists of a polydiene, such as polybutadiene, such as polybutadienestyrene, polyisoprene or butyl rubber. Particular mention is made of polybutadiene due to its chemical and mechanical properties making it particularly suitable for employment in a cell culture device.

Moreover, in non-limiting examples in which the surfactant molecules, e.g. the hydrophobic moiety thereof, comprise an unsaturated carbon-carbon bond, the unsaturated carbon-carbon bonds included in the polybutadiene can provide sites at which the surfactant molecules can be grafted, for example covalently bonded, to the polybutadiene second polymer.

It is noted that the lack of unsaturated carbon-carbon bonds in the case of, for example, a polyurethane second polymer may mean that this particular second polymer may not form crosslinks with surfactant molecules comprising an unsaturated carbon-carbon bond.

In some embodiments, the second polymer may comprise an unsaturated carbon-carbon bond, e.g. a vinyl functional group, for example as in the case of polybutadiene, and/or a hydride functional group, for example as in the case of a polysiloxane, and the at least some of the surfactant molecules each comprise an unsaturated carbon-carbon bond in the hydrophobic moiety. e.g. as in the case of the above-mentioned unsaturated surfactant, such as the unsaturated fatty acid. In such a non-limiting example, the crosslinking may be via a reaction between the unsaturated carbon-carbon bond of the surfactant and the unsaturated carbon-carbon bond, e.g. vinyl functional group, and/or hydride functional group of the second polymer.

The solvent can be any solvent in which the second polymer and the surfactant can be dissolved and/or dispersed in order to enable providing 104 of the solution 204 on the first surface 201, but which can also be evaporated in a subsequent step of the method 100. The solvent may also not or only minimally solvate the first surface 201 of the first polymer 200 on which the solution 204 is provided so that the polymer membrane can be formed on the first surface 201.

In at least some embodiments, the solvent comprises or consists of one or more of a hydrocarbon solvent, such as heptane, an alcohol solvent, such as isopropylalcohol, and a ketone solvent, such as acetone. For example, the solvent comprises or consists of heptane, for example when the second polymer comprises or consists of a polydiene, such as polybutadiene.

Where polybutadiene is used as, or included in the second polymer, the polybutadiene may be made using any suitable polymerization process. Particularly preferred is a polybutadiene formed in a Nd or Li-catalyzed polymerization reaction, as it is well-known per se that such polybutadiene has a low degree of branching (typically below 5%) and a low degree of polydispersity (Mw/Mn) of around 2. Li-catalyzed polybutadiene is particularly preferred due to its high degree of 1.2-vinyl content (about 11%). However, other types of polybutadiene. e.g. polybutadiene obtained from a Co, Ni or Ti-catalyzed polymerization reaction may be used instead.

Where a polydiene such as polybutadiene is used as the second polymer, the above-described crosslinking reaction between the second polymer and the surfactant molecules, e.g. unsaturated fatty acid, may be catalyzed using a peroxide catalyst (e.g. provided in addition to UV radiation and/or heating). Any suitable peroxide can be used to accelerate curing/crosslinking of the polydiene, e.g. polybutadiene, such as di-cumyl peroxide.

In another advantageous embodiment, a Li-catalyzed polybutadiene is used having a degree of branching in a range of 5-15% as it has been found that when the degree of branching of the polybutadiene is in this range, the reactivity of the polybutadiene with the unsaturated fatty acid can be improved. It is noted for the sake of completeness that it is well-known per se how to control the degree of branching when synthesizing polybutadiene such that this will not be further explained for the sake of brevity only.

Another class of second polymer that is specifically mentioned is silicones, in other words polysiloxanes. Such silicones may comprise a polydimethyl siloxane (PDMS) backbone including vinyl moieties to facilitate crosslinking through Pt-catalyzed addition reactions, e.g. with (poly methyl) hydrogen siloxanes. Instead of or in addition to such crosslinking with (poly methyl) hydrogen siloxanes, the vinyl-functionalized PDMS backbone may be crosslinked with the surfactant, e.g. a surfactant comprising or in the form of an unsaturated fatty acid. Such crosslinking reactions preferably involve hydrosilylation across the vinyl functionalities. Other carbon-carbon double bonds than the vinyl bonds may be used in such hydrosilylation reactions such as acetylenic bonds.

Curing of the polysiloxane may, for example, be initiated after mixing two parts: part A comprising a siloxane and a platinum catalyst and part B comprising the crosslinker.

In some non-limiting examples, after adding and mixing the surfactant the curing reaction may be slowed down. There accordingly may be a delay/idle time in which to provide the solution 204 onto the first polymer 200, e.g. via dispensing, spin coating etc. By subsequently heating the solution 204, for example up to a temperature in the range of 80-120° C., the curing may accelerate, for instance such that the curing is complete in less than 1 hour.

More generally, the cured/crosslinked polysiloxane may be formed from a multi-part starting material as previously explained in order to prevent premature crosslinking of the polysiloxane. Such multi-part starting materials are well-known per se: for example, such multi-part starting material kits are marketed by Wacker Chemie AG from Munich, Germany under the tradename Elastosil.

Such silicones may be formed by a crosslinking reaction between a vinyl-functionalized linear or branched silicone monomer or oligomer, e.g. a T-branched or Q-branched silicone monomer or oligomer and a linear hydride-functionalized silicone monomer or oligomer, between a hydride-functionalized linear or branched silicone monomer or oligomer, e.g. a T-branched or Q-branched silicone monomer or oligomer and a linear vinyl-functionalized silicone monomer or oligomer or mixtures of vinyl-functionalized and hydride-functionalized linear or branched silicone monomers or oligomers, e.g. a T-branched or Q-branched silicone monomer or oligomer and/or a mixture of linear hydride-functionalized and vinyl-functionalized silicone monomer or oligomer.

For example, a two-component silicone may be formed by a crosslinking reaction between a linear component 1 and a linear component 2, which each may correspond to the general formula below:

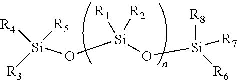

In component 1, R₁ and R₂ are individually selected from C₁-C₃ alkyl, R₃-R₈ are individually selected from C₁-C₃ alkyl and vinyl, with the proviso that at least one of R₃-R₅ and at least one of R₆-R₈ is vinyl. Preferably, at least three of R₃-R₈ are vinyl. In all embodiments, n may be in the range of 100-200,000. In a specific embodiment of component 1, each of the groups R₁-R₈ that is an alkyl group is a methyl group, i.e. the component 1 is vinyl-functionalized PDMS having terminal vinyl groups.

In component 2, R₁ is hydrogen, R₂=C₁-C₃ alkyl and R₂-R₈ are individually selected from C₁-C₃ alkyl or hydrogen, with the provision that at most one of R₃-R₅ and at most one of R₆₋₈ is hydrogen, and n may have any suitable value, such as n=3-1,000 or more specifically n=3-10. In a specific embodiment of component 2, none of the groups R₃-R₈ are hydrogen. In another specific embodiment of component 2, each of R₂-R₈ are methyl.

Component 2 typically acts as a crosslinking agent for component 1. Such agent crosslinks via crosslinking reactions of the hydrosylilation type, which are typically Pt-catalyzed, and are well-known per se, see for example WO 2009/147602 A2 and are therefore not explained in further detail for the sake of brevity. Several types of crosslinking agents and several types of Pt based catalysists are known per se, will not be further described herein and may be used to effect in the current method.

Any suitable silicone elastomer may be used. It should be understood that when crosslinking such silicones in the presence of the surfactant, e.g. an unsaturated fatty acid, the covalent bond between the silicone and the surfactant alternatively may be formed by a reaction between a hydride functional group of the (crosslinked) silicone and the unsaturated fatty acid as suggested in the below reaction mechanism:

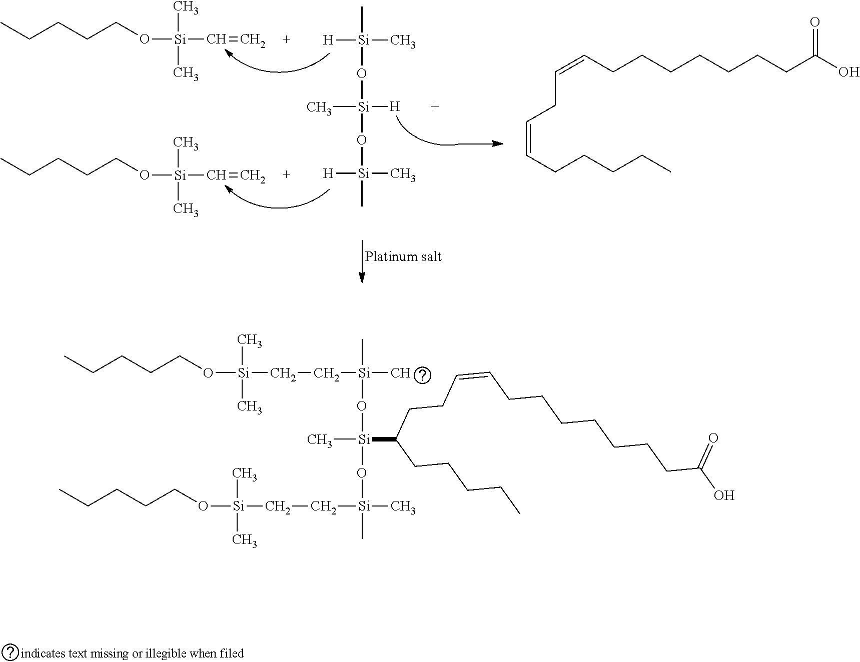

In order to prevent catalyst inhibition by the unsaturated fatty acid surfactant during such crosslinking reactions, the concentration of the unsaturated fatty acid may be limited, e.g. to a concentration of 1 wt. % or lower based on the solution 204. It has been found that below 1 wt. %, the fatty acid, e.g. linoleic acid, may not or only negligibly inhibit the crosslinking reactions, and curing and crosslinking at relatively high temperatures, e.g. higher than 80° C., can be achieved in spite of the presence of the fatty acid. Moreover, recipes based on such relatively low concentrations of the fatty acid, e.g. linoleic acid, have been found to work very well in terms of providing a polysiloxane-comprising or polysiloxane-based polymer membrane having polar moieties available at the surface of the polymer membrane.

In some non-limiting examples, a silicone backbone such as a (poly methyl) hydrogen siloxane backbone may be crosslinked with rubber-like polymers such as polybutadiene and polyisoprene, with the unsaturated fatty acid being incorporated in such a crosslinked product. This, for example, may be done to tune the water permeability of the final polymer membrane 206 as silicones typically have a rather open structure whilst such rubber-like polymers have a rather closed structure, such that the openness, i.e. the water permeability, of the crosslinked product can be tuned by the ratio of silicone and rubber-like polymer therein.

In this manner, the properties of the material according to this embodiment may be optimized for cell/protein interaction to build good scaffolds. For example, where the polymer membrane is a silicone-based, e.g. PDMS-based, a material that is highly permeable to water and other compounds (e.g. drugs) is obtained that facilitates maximized perfusion of these compounds into the cells on the polymer membrane. However, where real-time monitoring of drug consumption by cells is desirable, a polydiene-based material such as a polybutadiene-based material that is non-permeable to water and drugs may be preferable, as the rate of perfusion of these compounds to the cells can be controlled using, for instance, the density of apertures through the polymer membrane.

Returning to FIGS. 1A and 1B, the method 100 further comprises evaporating 106 the solvent while the solution 204 is on the first surface 201 to provide the polymer membrane 206. The thus formed polymer membrane 206 has a second surface 207 in contact with the first surface 201 of the first polymer 200, as schematically depicted in FIG. 1A.

The polymer membrane 206 can have any suitable thickness, depending, for example, on the manner in which the solution 204 is provided to the first surface 201 of the first polymer 200 and/or the intended function of the polymer membrane 206, e.g. in a fluidic device, such as a cell culture device.

Preferred thickness is less than 100 μm, less than 80 μm or less than 60 μm or less than 40 μm. Such membranes are generally fragile and benefit from the support layer in the form of the first polymer during manufacturing and or handeling.

In some embodiments, the polymer membrane 206 has a thickness less than 100 micrometers, e.g. in a ranged of 0.5 to 100 μm, such as in a range of 0.5-60 μm, or such as 5-40 μm, for example 20-60 μm, e.g. about 40 μm.

The thickness of the polymer membrane 206 can be controlled, for instance, via the manner in which the solution 204 is provided to the first surface 201 of the first polymer 200. For example, dispensing the solution 204 onto the first surface 201 can enable fabrication of a polymer membrane 206 having a thickness of 10-60 μm, whilst spin coating the solution 204 on the first surface 201 can permit a polymer membrane 206 having a thickness of 0.5-10 ∪m to be made.

It is also noted that polymer membranes 206 suitable for, for instance, culturing spheroids, organoids or biopsies thereon typically have a thickness in the range of 20-60 μm, e.g. about 20, or 40 μm.

Limiting the thickness of the polymer membrane 206, e.g. to less than or equal to e.g. 100 or 60 μm or other thicknesses indicated hereabove may, in some examples, provide optical transparency to enable microscopy such as for example confocal microscopy to be used to monitor cell culture through the polymer membrane 206 in examples in which the polymer membrane 206 is employed in a cell culturing device.

More generally, at least the first surface 201 of the first polymer 200 has greater affinity for the polar moiety than the second polymer.

By at least the first surface 201, and in some examples the entirety, of the first polymer 200 having greater affinity for the polar moiety than the second polymer, a preponderance of the surfactant molecules at the second surface 207 of the polymer membrane 206 may be orientated such that the polar moiety is made available at the second surface 207.

A higher affinity of the first polymer 200, or at least the first surface 201 of the first polymer 200, relative to the second polymer for the polar moiety can be achieved by selecting a first polymer 200 whose polarity at least at the first surface 201 is higher than that of the second polymer. Alternatively or additionally, the first polymer 200 may be selected to form stronger intermolecular interactions, such as hydrogen bonds, with the polar moiety at the first surface 201 than the second polymer.

Thus, the higher polarity of the first polymer 200 (at least) at the first surface 201 relative to the polarity of the second polymer can mean that the surfactant molecules orientate such that the polar moiety interacts, e.g. via permanent dipole-permanent dipole-type interactions, with the first surface 201 of the first polymer 200.

In embodiments in which the surfactant molecules each comprise a hydrophobic moiety, e.g. a hydrophobic tail moiety, the lower polarity of the second polymer relative to (at least the first surface 201) of the first polymer 200 further means that the hydrophobic moiety, e.g. hydrophobic tail moiety, preferentially interacts with the second polymer, thereby to assist this orientation of the surfactant molecules to make the polar moiety available at the second surface 207 of the polymer membrane 206.

By the polar moiety of the surfactant molecules being made available at the second surface 207 of the polymer membrane 206 in this manner, the surface properties of the second surface 207 may be particularly suitable for binding of, for example, a cell culturing protein, such as fibronectin, thereto. Alternatively, or additionally such second surface may allow cell culturing without first binding a cell culturing protein.

Such binding can, in at least some examples, comprise covalent bonding of the cell culturing protein to the polar moiety. In a non-limiting example, the polar moiety is a carboxylic acid group. In such an example, fibronectin can be covalently bonded to the polymer membrane via a reaction with the carboxylic acid group.

A well-documented binding protocol for covalently binding fibronectin to surface carboxylic acid groups using 1-ethyl-3-(3-dimethylamino) propyl carbodiimide hydrochloride (EDC) and N-hydroxysuccinimide (NHS) is given below:

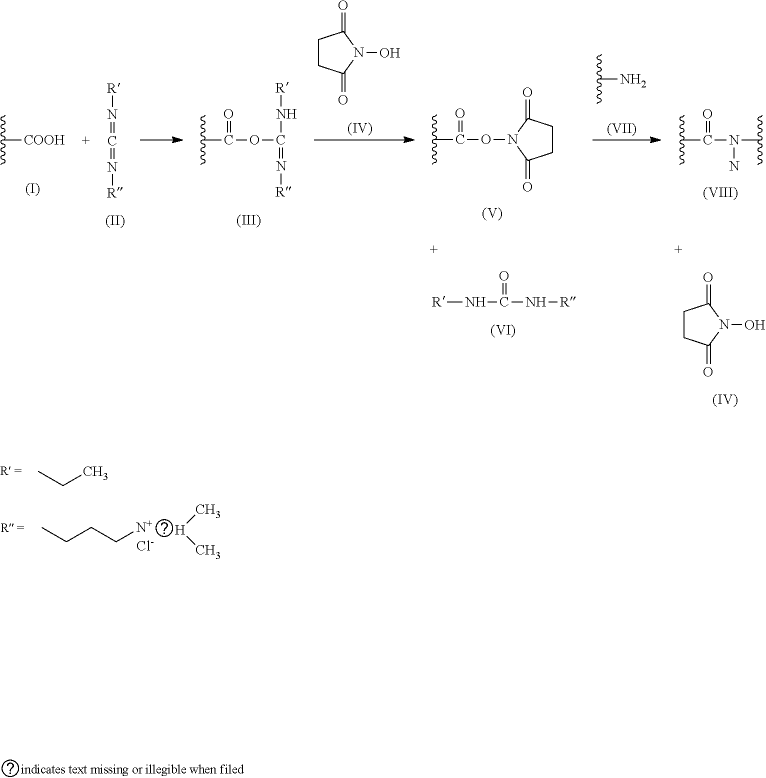

A description of this protocol can be found in: L. H. H. Olde Daminnk, P. J, Dijkstra, M. J. A. Luyn, P. B. v. Wachem, P. Nieuwenhuis, and J. Feijen, “Cross-linking of dermal sheep collagen using a water-soluble carbodiimide”, Biomaterials, 17 (8) pp. 765-774 (1996).

Moreover, due to the first polymer 200 being employed for effecting the above-described orientation of the polar moiety, the polymer membrane 206 can be relatively straightforwardly released therefrom, for instance by peeling the first polymer 200, e.g. first polymer foil, and the polymer membrane 206 from each other, and/or by dissolving the first polymer 200 in a solvent in which the polymer membrane 206 is less soluble or insoluble so as to separate the polymer membrane 206 from the first polymer 200. This provides a more convenient method 100 of fabricating a polymer membrane 206 having polar moieties available at a surface thereof.

In some embodiments, the first polymer 200 comprises or consists of one or more of polyvinylalcohol, polyurethane and polyethylene glycol. Such first polymers 200, e.g. in the form of a foil, can be releasable from the polymer membrane 206, e.g. by peeling and/or dissolving of the first polymer 200.

Particular mention is made of polyvinylalcohol and polyethylene glycol because the water-solubility of such first polymers 200 means that water can be used to remove the first polymer 200 from the polymer membrane 206.

It is noted that in the case of, for example, a polyurethane first polymer 200, the relatively low solubility of polyurethane may mean that peeling or stripping of the polyurethane first polymer 200 from the polymer membrane 206 is used instead of dissolving.

Moreover, such a polyvinylalcohol, polyurethane or polyethylene glycol first polymer 200 may be more polar and/or form stronger intermolecular interactions, such as hydrogen bonds with, and thus have greater affinity for, the polar moiety than the second polymer, for example when the second polymer is a polydiene, such as polybutadiene.

Employing the first polymer 200 may have further advantages, for example relating to manufacturability. Returning to FIGS. 1A and 1B, the method 100 may comprise cutting 108 a laminate comprising the polymer membrane 206 layered on the first polymer 200 to a desired shape and/or size. Such cutting 108 is enabled by it being a cuttable (first) polymer 200 which is employed to orientate the surfactant molecules such as to make the polar moieties available at the second surface 207 of the polymer membrane 206. This cutting step is optional.

Referring to FIG. 1B, the method 100 may comprise separating 110 the polymer membrane 206 and the first polymer 200. Such separating 110 can be implemented in any suitable manner. For example, the separating 110 can comprise delaminating, e.g. peeling, the first polymer 200 from the polymer membrane 206 and/or by dissolving the first polymer 200 in a solvent in which the polymer membrane 206 is less soluble or insoluble so as to separate the polymer membrane 206 from the first polymer 200, as previously described.

In the non-limiting example shown in FIG. 1B, the frame 202 is also separated, e.g. delaminated, from the first polymer 200 together with the polymer membrane 206, while the frame 202 continues to hold the polymer membrane 206.

In some embodiments, the second polymer is optically transmissive, e.g. optically transparent, thereby to permit optical interrogation of, for instance, cells located on the other side of the polymer membrane 206 with respect to the apparatus, e.g. microscope, used for the optical interrogation.

In some embodiments, such as in the exemplary method 100 shown in FIGS. 1A and 1B, one or more grooves and/or apertures 210 are formed in the polymer membrane 206 in step 114. Such groove(s) and/or aperture(s) 210 may assist the polymer membrane 206 to fulfil a function in, for instance, a cell culture device, such as enabling nutrients to be supplied and waste products to be removed from the cells via the groove(s) and/or aperture(s) 210.

The size/diameter of the apertures 210 can be selected according to what is most practical, e.g. for handling and/or microscopy. Such aperture(s) may, for example, have a diameter in the range of 1-1000 μm. Their shape may be rectangular, square, but preferably is rounded such as circular. The apertures preferably are provided in a regular array (e.g. square and/or hexagonal patterns) of apertures over at least a part of the area of the membrane. The spacing of apertures may be chosen according to for example desired permeability of the membrane, where an increasing number apertures per unit area may relate to an increasing permeability.

For certain applications, such as when cell layers are desired to grow on the polymer membrane 206, the one or more aperture(s) 210 may be smaller than 8 μm in diameter to order to retain cells on the correct side of the polymer membrane 206, for instance to avoid or minimize cells crossing over, e.g. when epithelial cells are at one side of the polymer membrane 206 and endothelial cells are on the other side.

For spheroids and organoids, the aperture(s) 210 may have a diameter in the range of 1 to 250 μm for example 50-250 μm. However, other ranges may be used such as for example 1 to 50 μm, more preferably a range of 1-10 μm or even 1 to 8 μm may be used, depending on the dimensions of the spheroids and organoids. The smaller dimensions generally allow passage of smaller molecules while cells or clusters of cells are less well able to pass the apertures.

When the cell culture device includes a fluidic system, it may be particularly important for spheroids and organoids that these are not unintentionally washed away by the flow and that they remain at their positions. Hence, the diameter of the aperture(s) 210 may be selected to retain such spheroids or organoids, as the case may be, on the intended side of the polymer membrane 206. The appropriate aperture diameter as mentioned herein before may be used for this.

For biopsies, the aperture(s) 210 may have a diameter in the range of 50-1000 μm. For example, biopsies can be 4 mm long, 0.5 mm wide, and 300 μm high.

For spheroids, organoids and biopsies the cell culture device may comprise a plurality of such polymer membranes 206, e.g. with a pair of such polymer membranes 206 opposing each other.

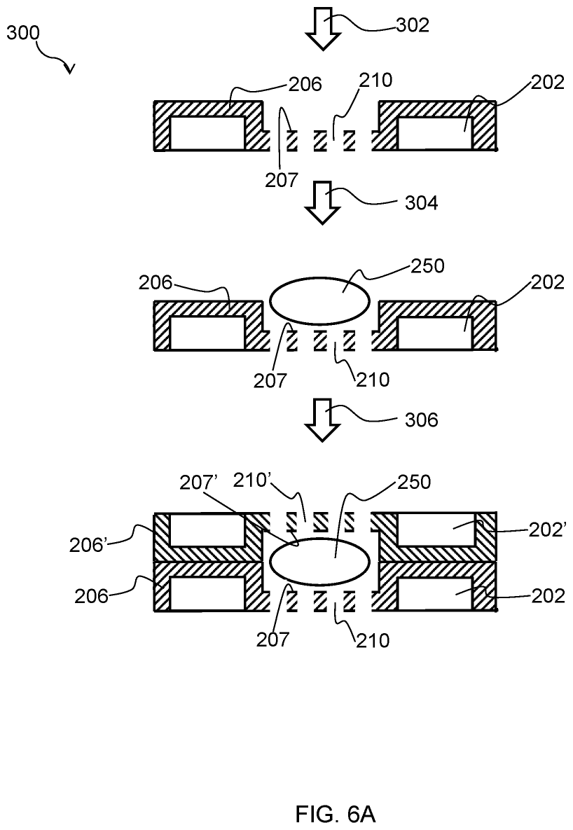

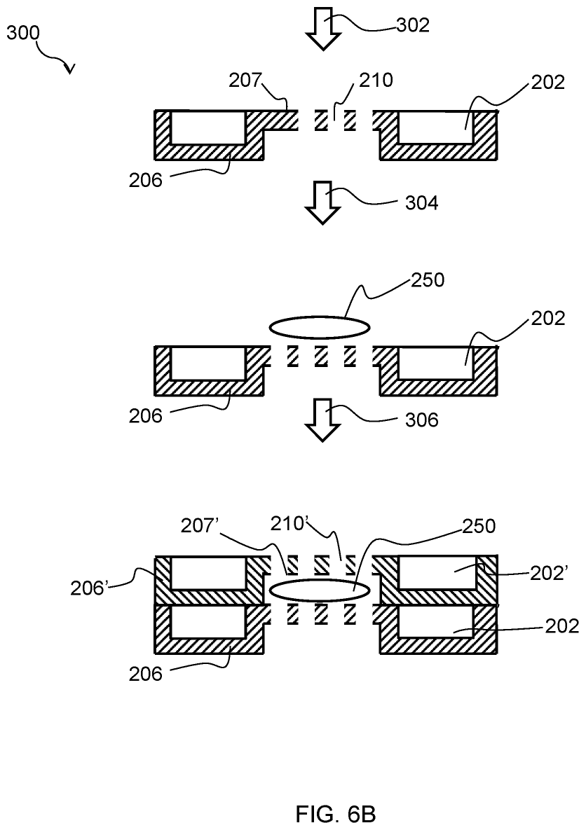

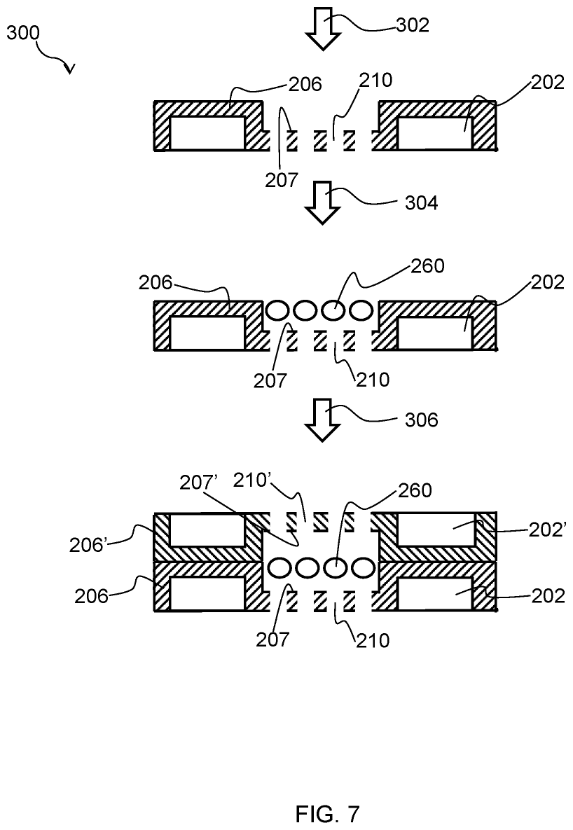

In a non-limiting example, which will be further explained herein below, cell culturing may take place on one of the polymer membranes 206, with another polymer membrane 206 functioning as a setter/keeper to allocate the spheroids, organoids or biopsy between the polymer membranes 206. Multiple configurations may be used as will be explained below with reference to for example FIGS. 6 and 7.

For monitoring growing, treatment and staining the spheroids, organoids and biopsies it may be important that they are fixed in position. Particularly for biopsies the history of geometry may be key because biopsies can have a tumor side and a healthy tissue side. Where treatments, such as chemotherapy treatments, are being evaluated in may be critical to observe how the tumor tissue and the healthy tissue respond.

In the non-limiting example shown in FIGS. 1A and 1B, such one or more grooves and/or apertures 210 are formed in the polymer membrane 206 via laser drilling, in other words laser drilling ablation process. In this particular example, the method comprises mounting 112 the polymer membrane 206 on a holder 208 to enable subsequent laser drilling of the grooves and/or apertures 210 in step 114. Exemplifying methods of optical or laser drilling apertures with diameters in desired ranges and in polymers have been disclosed in for example US2014/0127744, which is incorporated by reference in its entirety.

Any suitable conditions can be employed for such laser drilling. The following is provided by way of a non-limiting example: wavelength: 1025 nm; pulse duration: 350 fs; average power: 500 mW; Spot size: 15˜20 μm; number of pulses: 25˜100. This is especially suitable for polybutadiene based polymer membranes. In another example suitable for drilling polysiloxan membranes the following conditions may be used: wavelength: 515 nm; pulse duration: 350 fs; average power: 13 mW (pulse energy 13 μJ and repetition rate 1 kHz); Spot size: 1˜20 μm; number of pulses: 5˜100.

The polymer membrane 206 can be mounted on the holder 208 via the frame 202, in other words while the polymer membrane 206 is attached to the frame 202, as shown.

Once the grooves and/or apertures 210 have been formed in the polymer membrane 206 via the above-described laser drilling process, the holder 208 may be removed in step 116.

In other non-limiting examples, an alternative technique for forming such grooves and/or holes 210 may be employed, such as for example by shaping the first surface 201 of the first polymer 200 such that such grooves and/or holes 210 are formed in the polymer membrane 206 following evaporation of the solvent. The first surface 201 may be provided with a relief structure where protrusions define the locations of future apertures. The dimensions measured in the plane of the surface may then define shape and size of such apertures. Exemplifying methods can be found in US20200360923 which is incorporated by reference in its entirety. Also, polymer membranes with indentations may be made using such method as described herein before. Apertures may be made in the areas of indentation of the polymer membrane.

In some embodiments, the method 100 further comprises functionalizing the second surface 207 of the polymer membrane 206 with a cell culturing protein. The polar moieties made available at the second surface 207 facilitate this functionalization, as previously described.

The cell culturing protein can comprise or consist of one or more of collagen, elastin, and fibronetin. In the case of fibronectin, this protein can be coupled, for example in the above-described manner using EDC and NHS, to a polar moiety in the form of a carboxylic acid group.

In non-limiting examples in which the first polymer 200 can be removed from the polymer membrane 206 by dissolving the first polymer 200, this process can be combined with protein coupling by including the protein and any coupling agents, such as EDC and NHS, in the same solvent used for dissolving the first polymer 200. For example, when water is used to remove, or assist in removing, a polyvinylalcohol first polymer 200, the protein coupling may be carried out using water as a solvent.

Other chemistry methods of bonding proteins to the surface may be used.

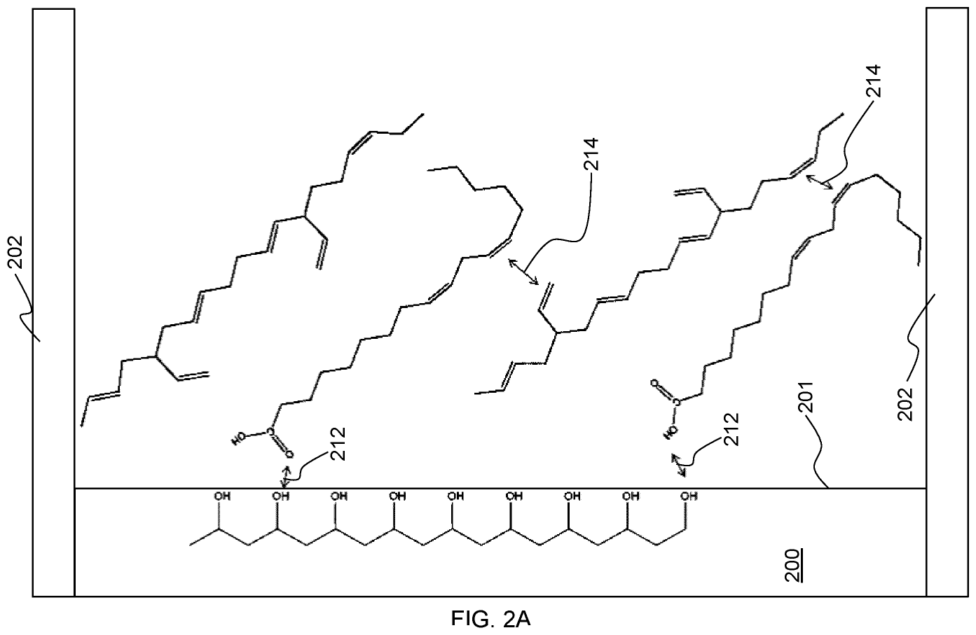

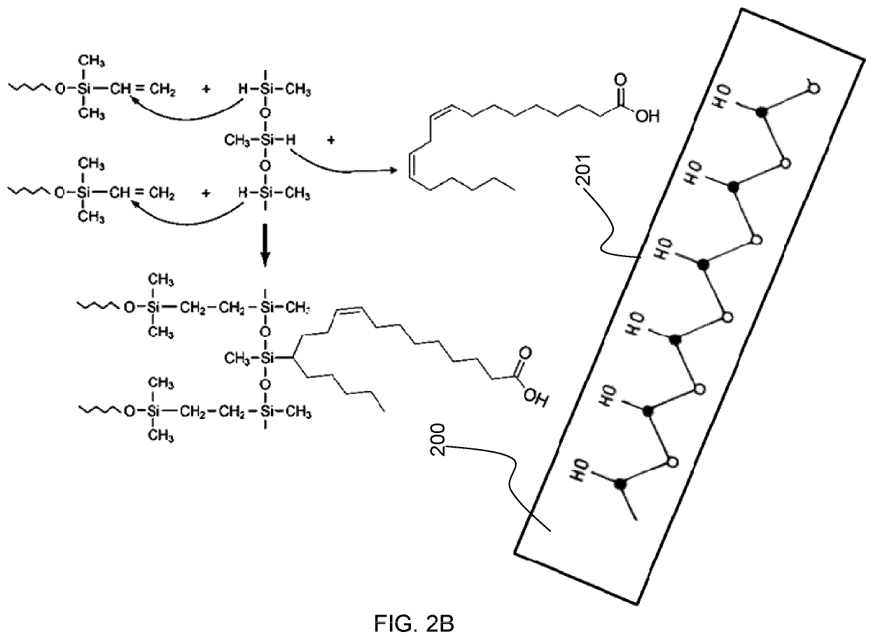

FIG. 2A schematically depicts, in a magnified side view which also shows the optional frame 202 and the surface 201 of the first polymer 200, orientating of the surfactant molecules. In this non-limiting example, the surfactant is linoleic acid, e.g. α-linolenic acid, and the orientating is such that a polar moiety thereof, in this case a carboxylic acid group, is made available at the second surface 207 of the polymer membrane 206, as previously described.

To this end a polar attraction, represented by the double-headed arrows 212, between the polar moiety of the surfactant and the first polymer, in this non-limiting example a polyvinylalcohol foil, assists to orientate the surfactant molecules such that the polar moiety is made available at the second surface 207 of the polymer membrane 206.

FIG. 2A thus shows how the polar moieties, in this case the carboxylic acid groups, are directed to, and interact with, the first polymer 200, in this case the first surface 201 of the polyvinyl alcohol foil, after dispensing of the solution 204.

In the non-limiting example schematically depicted in FIG. 2A, the second polymer has unsaturated carbon-carbon bonds (alkenyl groups), including vinyl groups, for crosslinking with unsaturated carbon-carbon bonds of the surfactant, as represented by e.g. the double-headed arrows 214. Although not shown, there may also occur crosslinking between carbon-carbon double bonds of different second polymer molecules.

In this specific example, such crosslinks are formed between the unsaturated carbon-carbon bonds, including vinyl groups, of the polybutadiene second polymer and the unsaturated carbon-carbon bonds of the linoleic acid surfactant.

FIG. 2B shows an example in which crosslinks are formed via reaction between silicon-hydride bonds and the unsaturated carbon-carbon bonds of an unsaturated fatty acid surfactant, in this case linoleic acid. A similar reaction between silicon-hydride bonds and unsaturated carbon-carbon bonds takes place between the siloxane components shown in FIG. 2B.

A polar attraction between the polar moiety, in this case the carboxylic acid group, of the surfactant and the first polymer, in this non-limiting example a polyvinylalcohol foil, assists to orientate the surfactant molecules such that the polar moiety is made available at the second surface 207 of the polymer membrane 206, as previously described.

FIG. 3 provides a plan view of a polymer membrane 206 and a frame 202. In this non-limiting example, the polymer membrane 206 is positioned in a cavity delimited by the frame 202. The frame can be part of the polymer membrane which is supported by the frame 202 at its edges.

The frame 202 can be formed of any suitably stiff material, such as a metal or metal alloy, e.g. stainless steel, or a plastic or polymer, e.g. a polymer which is insoluble in the solvent(s) used in the above-described steps of the method 100. Examples of such a frame polymer are polyether ether ketone and a fluoropolymer, such as Teflon®.

The dimensions and shape of the frame 202 can be selected according to the intended application, e.g. in a fluidic device, such as a cell culture device. In some embodiments, the frame 202 is also included, together with the polymer membrane(s) 206 in such a device.

The diameter 216 of the cavity can be, for example, in a range of 1-10 mm, such as 4-5 mm. The minimum width 218 of the frame 202 from the edge of the cavity to the outer edge of the frame 202 may be 1 mm.

The cavity can have any suitable shape, such as circular. Similarly any suitable overall frame shape can be considered. A rectangular/square frame 202 is shown in FIG. 3.

In the non-limiting example shown in FIG. 3, the diameter of the cavity is 5-6 mm, and the frame has an overall square shape measuring 10 mm by 10 mm.

Further evident in FIG. 3 are apertures 210 formed in the polymer membrane 206, e.g. via the above-mentioned laser drilling process.

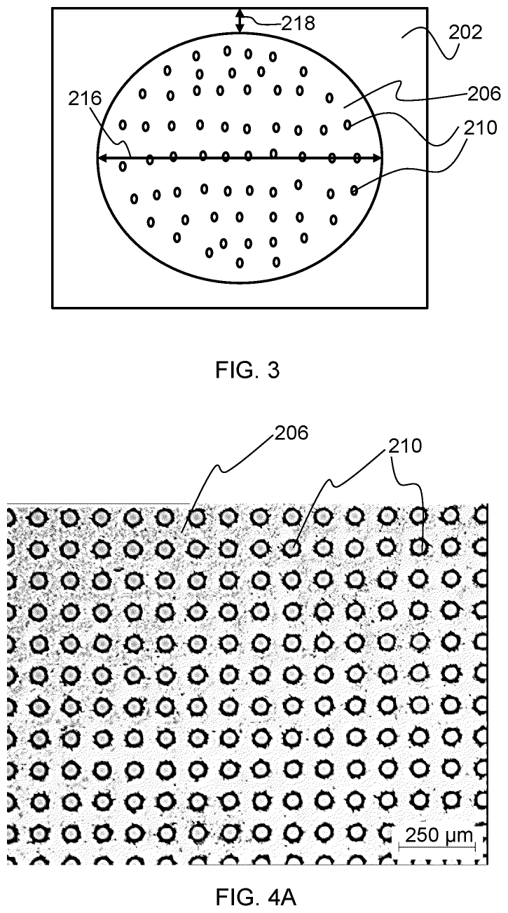

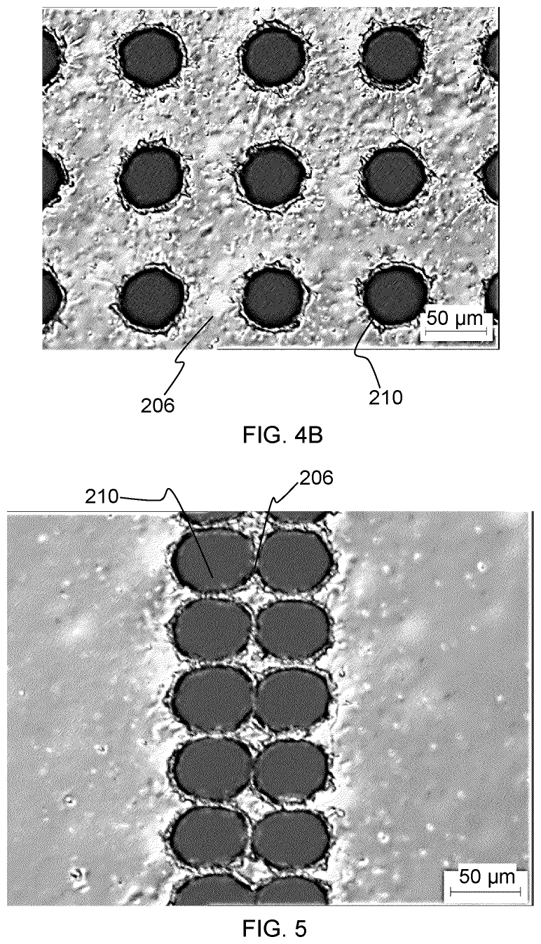

FIGS. 4A and 4B provide micrographs of part of an exemplary polymer membrane 206 having apertures 210 formed via laser drilling.

FIG. 5 provides a micrograph of part of another exemplary polymer membrane 206 having apertures 210 formed via laser drilling where minimum polymer membrane material is left between the apertures 210. This can be used to provide membranes with increased porosity.

Further provided is a fluidic device comprising one or a plurality of the polymer membrane 206. FIGS. 6A and 6B schematically depict encapsulation of a tissue sample 250 by a plurality of polymer membranes 206, 206′. FIGS. 7A and 7B schematically depict encapsulation of spheroids/organoids 260 by a plurality of polymer membranes 206, 206′.

More generally a fluidic device, e.g. a cell culturing device, is provided which fluidic device comprises: a polymer membrane 206 and a further polymer membrane 206′, the polymer membrane having a first surface 207 which opposes a second surface 207′ of the further polymer membrane 206′, at least one of the polymer membranes 206, 206′ comprising a surfactant comprising surfactant molecules each having a polar moiety at the respective surface or surfaces.

One or both of the surfaces 207, 207′ may be functionalized with a cell culturing protein. Such functionalizing may be via the polar moiety, e.g. via covalent bonding of the cell culturing protein to the polar moiety, as previously described.

One, or preferably both, of the polymer membranes 206, 206′ may comprise one or more grooves and/or apertures 210, 210′, as shown in FIGS. 6A, 6B and 7.

One or both of the polymer membranes 206, 206′ may be fabricated, for example, according to any embodiment of the above-described method 100.

There is also provided a cell culturing method 300 comprising encapsulating cells with at least one of the polymer membrane 206 according to the present disclosure, for example between the above-described first and second polymer membranes 206, 206′.

Referring to FIGS. 6A and 6B, such a method 300 may comprise providing 302 a polymer membrane 206 comprising a surfactant comprising surfactant molecules each having a polar moiety at a surface 207 of the polymer membrane 206, the surface 207 being preferably functionalized with a cell culturing protein.

Cells may then be provided, e.g. dispensed, 304 on the surface 207 of the polymer membrane 206.

In the non-limiting example depicted in FIG. 6, the cells comprise or take the form of a tissue sample or biopsy 250. Alternatively, the cells comprise or take the form of spheroids/organoids 260, as shown in FIG. 7. They may also be single cells.

The method 300 further comprises providing 306 a further polymer membrane 206′. A further surface 207′ of the further polymer membrane 206′ opposes the polymer membrane 206, with the cells 250, 260 being thus encapsulated between the surfaces 207, 207′ of the polymer membranes 206, 206′.

In some embodiments, the further polymer membrane 206′ comprises a surfactant comprising surfactant molecules each having a polar moiety at the further surface 207′, the further surface 207′ being preferably functionalized with a cell culturing protein, e.g. such that both of the opposing surfaces 207, 207′ are functionalized with a cell culturing protein. In other embodiments, the further polymer membrane 206′ does not comprise a surfactant comprising surfactant molecules each having a polar moiety at the further surface 207′. For example when the further polymer membrane is the same as the polymer membrane 206 and stacked back to front as in FIG. 6B. A similar stacking and polymer membrane variation may be envisaged for the embodiments of FIG. 7.

Note that the polymer membranes 206 with the surface 207 in its recess may be made with a process as describe herein e.g. with regard to the FIGS. 1A and 1B using a relief first polymer having protrusions corresponding to the recess.

One, or preferably both, of the polymer membranes 206, 206′ may comprise one or more grooves and/or apertures 210, 210′. The groove(s) and/or aperture(s) 210, 210′ may assist nutrients to be supplied and waste products to be removed from the cells via the groove(s) and/or aperture(s) 210, 210′, and, in some examples, enable monitoring of the cell sample 250, 260, as previously described. Such aperture(s) 210 may, for example, have a diameter in the range of 1-1000 μm.

One of the polymer membranes 206, 206′ can be regarded as a carrier membrane, and the other of the polymer membranes 206′, 206 can be regarded as an encapsulation membrane. In such an example, neighboring apertures 210 of the encapsulation membrane 206 may be closer to each other than neighboring apertures 210′ of the carrier membrane 206′. Alternatively or additionally, the apertures 210 of the encapsulation membrane 206 may have a larger diameter than the apertures 210′ of the carrier membrane 206.

In many cases for cell growing, cell proliferation, cell growth to spheroids and organoids 260 and after placing a biopsy 250, it is undesirable for a cell to be able to pass through the polymer membrane 206. In this case, the sample 250, 260 may be placed on a polymer membrane 206 having apertures 210 with a diameter of, for example, 5-7 μm.

In examples in which it is desired to grow a double layer with epithelial cells on top of the polymer membrane 206 and endothelial cells on the other side of the polymer membrane 206, a key requirement is that the epithelial and endothelial cells do not become mixed up. Hence, the apertures 210 may be sufficiently small, e.g. 5-7 μm, to prevent cells from passing through the polymer membrane 206. In this way, the double layer of cells can grow and differentiate to an inner and an outer layer, e.g. to mimic a gut, lung or skin.

In examples in which it is desired only to hold spheroids and organoids 260 in place (e.g. with a geometry fixed for studying its growth and responses to a drug by using markers and staining) between two polymer membranes 206, 206′, apertures 210, 210′ can be provided in both polymer membranes 206, 206′ smaller than the diameter of the spheroids/organoids.

In examples in which it is desired to fix the geometry of a biopsy 250, the polymer membrane 206 can be selected for cell permeability or non-cell permeability (depending on the needs, restrictions for the assay). The biopsy 250 can, for example, be kept in place in a moving fluid with a covering polymer membrane 206′ on top with bigger apertures 210′ which are more closely spaced to each other. A covering polymer membrane 206′ with apertures 210′ large enough to encapsulate, fix a biopsy 250 and relatively close to each other (in other words a relatively “open” polymer membrane 206′) may be favorable for optical analysis via microscopy, e.g. due to such an open polymer membrane 206″ facilitating focusing. Less material in the polymer membrane 206′ may mean that more optical clearance is provided for imaging.

In some examples, polymer membranes 206 can be stacked, e.g. to bring more different organoids 260 together in one fluidic device, e.g. one organ-on-chip device. For instance, a liver organoid can stacked with a pancreas organoid between three membranes so that they can live/communicate/exchange proteins with each other.

In a further example, the metastasis of cancer cells coming from a cancer biopsy may be studied. In such an example, the diameter of the apertures 210 can be used to select whether or not cells are let through the polymer membrane 206 and thus filtered into an adjacent chamber where they can be collected, e.g. for imaging.

Hence, more generally, a fluidic device may comprise two polymer membranes 206, 206″ whose apertures 210, 210′ have different sizes relative to each other, with a first chamber defined between the polymer membranes 206, 206′ and a second chamber separated from the first chamber by one of the polymer membranes 206, 206″.

It is further noted that the polymer membranes 206, 206′ may be brought together using the frames 202, 202′. Thus, in such a non-limiting example, the frames 202, 202′ may become parts of the fluidic device, e.g. cell culturing device, by which the polymer membranes 206, 206′ are mounted.

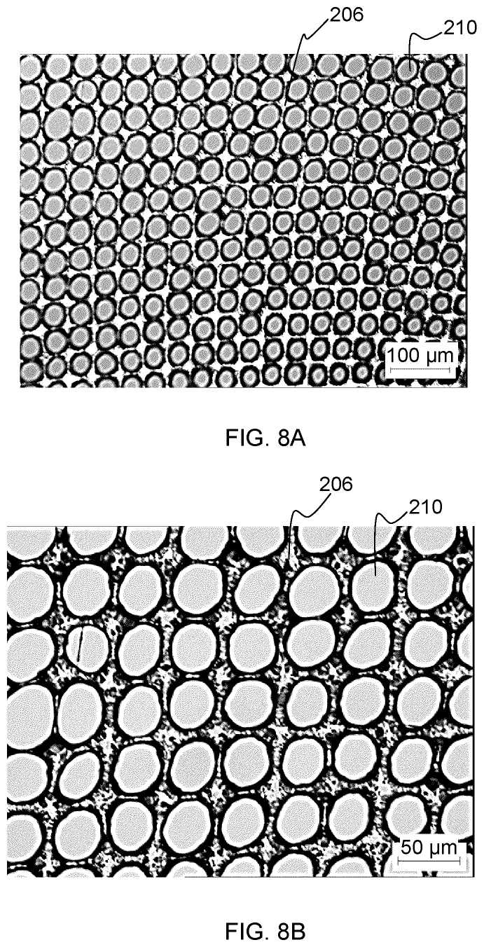

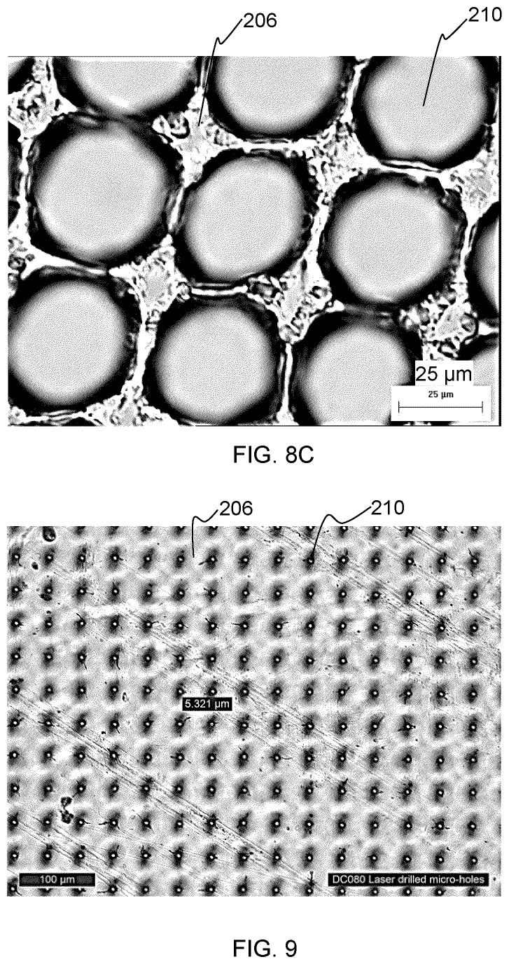

FIGS. 8A to 8C provide micrographs of an exemplary polymer membrane 206 for cell sample encapsulation, e.g. for use in the method 300 or cell culturing device described herein or above.

FIG. 9 provides a micrograph of an exemplary linoleic acid-modified polysiloxane polymer membrane 206, formed in the manner depicted in FIG. 2B, having laser-drilled apertures 210 each with a diameter of 5-7 μm. This polymer membrane 206 is permeable for medium, proteins and drugs, but not for cells, since cell cores typically measure 8 μm across.

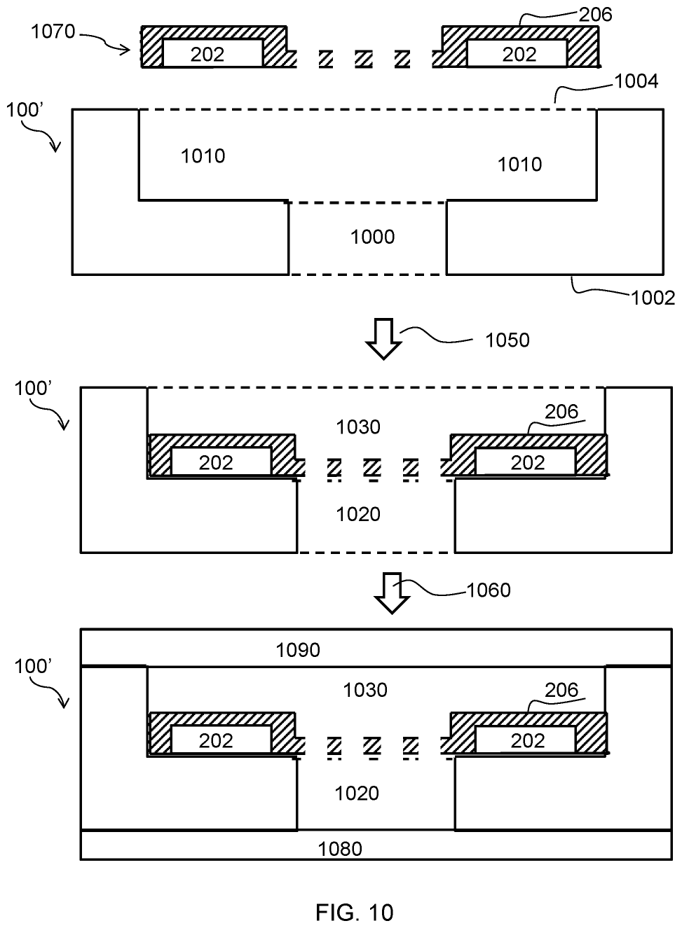

FIG. 10 provides a micrograph of an exemplary polybutadiene

A flexible soft membrane was fabricated by dispensing 104 a mixture of polybutadiene and unsaturated linoleic acid dissolved in heptane over a frame 202 onto a relatively polar polyvinylalcohol foil 200; carboxylic acid groups of the linoleic acid being directed to the surface 201 of the foil 200. After evaporating 106 the heptane and removal 110 of the foil, the resulting polymer membrane 206 was found to be straightforwardly mountable/incorporatable in a fluidic device, e.g. a “3D” fluidic device. The surface of the polymer membrane 206 has polar moieties, in this example carboxylic acid groups, where covalent bonds with proteins, e.g. human proteins, are possible. It is noted that such surface carboxylic acid groups can be detected using, for example, Oregon green 488 Cadaverin. Methods for functionalising such surfaces to make them suitable for cell culturing or using such surfaces directly (e.g. without functionalising them) for cell culturing have been disclosed herein and in for example WO2019015988 and WO2021058657.

A polysiloxane membrane was fabricated by dispensing 104 a layer of a mixture of Elastosil RT604™ (procured from Wacker), which is a mixture of a polysiloxane bearing vinyl end groups, a poly methylhydrogen siloxane and hydrosililation catalyst, and linoleic acid on a foil of polyvinyl alcohol. The dispensing may be done using isopropanol and or heptane as the solvent of the mixture. The concentrations in such solvent can be adjusted to change viscosity of the mixture. Lower viscosity layers can be used for depositing thinner layers of the mixture resulting in thinner polymer membranes. In this example no solvent was used. The applied layer was cured at room temp for an hour after which the polyvinly alcohol was removed by dissolution in water. If shorter curing times are needed, other types of castable Elastosils™ can be used.

It is noted, in general, that the methods, polymer membranes and fluidic devices of the present disclosure are applicable in all kind of carriers for cell culturing and for any oncology tests in fluidic systems.

For the purpose of describing the manufacturing of cell culturing and/or fluidic devices according to the current disclosure, an adjusted design and manufacturing method as disclosed in e.g. WO2019015988 may be used as an example. In particular the therein provided description pertaining to the therein disclosed FIGS. 3 to 8 for manufacture of parts and assembly of parts may be slightly adjusted according to the following description.

Thus, referring to for example FIG. 6 of WO2019015988, the fluidic module part 100 may be slightly adjusted in design. The adjusted manufacture and design is represented by steps and crossections in FIG. 10 of the current disclosure. Using the methods disclosed in WO2019015988 a fluidic module 100′ is manufactured. The fluidic module 100′ may for example be made of an elastomer such as polydimethylsiloxane, preferably without any of the carboxylic acid modifications as described in WO2019015988. It may be molded as described in WO2019015988.

At this first stage in FIG. 10. i.e. before step 1050, the fluidic module 100′ comprises a cavity 1000 (in this case cylindrical) extending from a first surface 1002 of the fluidic module to a second surface 1004 of the fluidic module. The cavity 1000 is meant to accommodate a polymer membrane according to the current disclosure (e.g. the polymer membrane 1070) after mounting of such membrane. For convenient mounting, the cavity may have, and in this case has, a recess extending from the surface 1004 along a chosen depth less than the thickness (measured as the distance between 1002 and 1004) into the fluidic module and towards the surface 1002 as represented by sections 1010. Such recess is however not always necessary.

Also shown in the first stage is a polymer membrane 1070 to be mounted to the fluidic module 100′. The polymer membrane 1070 can be one that results from step 116 in FIG. 1B having a polymer membrane layer supported by a frame 202. Between the shown sections of the frame 202 is a porous part of the membrane.

The cavity 1000 (including any recesses 1010), is designed such that upon mounting the polymer membrane 1070 to the fluidic module 100′ at least a part of that cavity 1000 becomes bound by the porous part of the polymer membrane. The polymer membrane is oriented such that the bound part of the cavity faces the second surface 207 (see FIG. 1B) of the polymer membrane 1070 so that the bound cavity part can serve as a cell culturing cavity.

Thus in step 1050, membrane 1070 is mounted to the fluidic module 100′ with its second surface directed downward in the FIG. 10, thereby dividing the cavity in a subcavity 1020 and a sub cavity 1030, where the subcavity 1020 corresponds to the part of the cavity 1000 for use with cell culturing. The membrane 1070 is designed to snugly fit into the recesses and since the fluidic module is elastic can be clamped by stretching the elastomer ever so slightly. The recess may be shaped according to the shape of the frame for a good fit and snug accommodation of the membrane. As can be seen from the second stage of the method of FIG. 10, the membrane now separates the cavity in the two subcavities 1020 and 1030 where the subcavity 1020 is in direct contact with the polymer membranes' second surface 207.

Although not shown in the cross sections in FIG. 10, the fluidic module 100′ is further designed as disclosed in WO2019015988 and accordingly comprises fluidic channels connected to the subchannels 1020 and 1030. Thus with reference to FIG. 6 2019015988 one of such channels is shown as part 115 and extends in one diagonal direction in the plane of the fluidic module. The other channel is on the bottom side of the fluidic module (hence cannot be seen in the perspective drawing of FIG. 6 of WO2019015988) and runs in the other diagonal direction in the plane of the fluidic module.

In step 1060 of the method of FIG. 10, cover plates 1080 and 1090 are attached to the surfaces 1002 and 1004 of the fluidic module 100′. The cover plate 1080 corresponds to the cover plate 210 of FIG. 6 in WO2019015988 and the cover plate 1090 to cover plate 220 of FIG. 6 in WO2019015988. Thus, the cover plates include all features FIG. 6 in WO2019015988 such as openings that connect to the fluidic channels and provide connections to the outside world for providing fluids to the channels as described in WO2019015988.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality.

The mere fact that certain measures are recited in mutually different dependent claims does not indicate that a combination of these measures cannot be used to advantage.

If the term “adapted to” is used in the claims or description, it is noted the term “adapted to” is intended to be equivalent to the term “configured to”.

Any reference signs in the claims should not be construed as limiting the scope.

### Further Examples

Example 1. A method (100) of fabricating a polymer membrane suitable for a cell culture device, the method comprising:
providing (104) a solution (204) on a first surface (201) of a first polymer (200), the solution comprising a second polymer, a surfactant, and a solvent, wherein the surfactant comprises surfactant molecules each having a polar moiety; and
evaporating (106) the solvent while the solution is on the first surface to provide said polymer membrane (206), the polymer membrane having a second surface (207) in contact with the first surface, wherein at least said first surface of the first polymer has greater affinity for the polar moiety than the second polymer.

Example 2. The method (100) according to example 1, wherein the second polymer comprises an elastomer and/or has a Shore A hardness of 70 or less.

Example 3. The method (100) according to example 1 or 2, wherein the second polymer comprises at least one selected from a polydiene, a polysiloxane, and a polyurethane: optionally wherein the second polymer comprises polybutadiene.

Example 4. The method (100) according to any of examples 1 to 3, wherein the first polymer (200) comprises one or more of polyvinylalcohol, polyurethane, and polyethylene glycol.

Example 5. The method (100) according to any of examples 1 to 4, wherein the solvent comprises one or more of a hydrocarbon solvent, an alcohol solvent, and a ketone solvent.

Example 6. The method (100) according to any of examples 1 to 5, wherein the polar moiety is a carboxylic acid group, an amine group or an amide group.

Example 7. The method (100) according to any of examples 1 to 6, wherein the surfactant comprises a fatty acid, the carboxylic acid group of the fatty acid defining the polar moiety.

Example 8. The method (100) according to any of examples 1 to 7, further comprising crosslinking the second polymer and at least some of the surfactant molecules: optionally wherein the second polymer comprises an unsaturated carbon-carbon bond and/or a hydride functional group, and said at least some of the surfactant molecules each comprise an unsaturated carbon-carbon bond, said crosslinking being via a reaction between the unsaturated carbon-carbon bond of the surfactant molecules and the unsaturated carbon-carbon bond and/or hydride functional group of the second polymer.

Example 9. The method (100) according to any of examples 1 to 8, comprising separating (110) the polymer membrane (206) and the first polymer (200).

Example 10. The method (100) according to example 9, wherein said separating (110) comprises peeling the first polymer (200) and the polymer membrane (206) apart from each other and/or dissolving the first polymer in a solvent in which the polymer membrane is less soluble or insoluble so as to separate the polymer membrane from the first polymer.

Example 11. The method (100) according to any of example s 1 to 10, comprising forming (114) one or more apertures and/or grooves (210) in the polymer membrane (206): optionally wherein the forming comprises laser drilling the polymer membrane.

Example 12. The method (100) according to any of example s 1 to 11, comprising functionalizing the second surface (207) with a cell culturing protein; optionally wherein the cell culturing protein comprises fibronectin.

Example 13. A polymer membrane (206) obtainable from the method according to any of examples 1 to 12.

Example 14. A cell culturing device comprising one or a plurality of the polymer membrane (206) according to example 13.

Example 15. A cell culturing device comprising a polymer membrane (206) and a further polymer membrane (206′), the polymer membrane having a surface (207) which opposes a further surface (207′) of the further polymer membrane, at least one of the polymer membranes comprising a surfactant comprising surfactant molecules each having a polar moiety at the respective surface or surfaces (207, 207′).

Example 16. A cell culturing method comprising immobilizing cells within a cell culturing device using at least one of said polymer membrane (206) according to example 13.

1. A method of fabricating a polymer membrane suitable for a cell culture device, the method comprising:
providing on a first surface of a first polymer:
a surfactant comprising surfactant molecules each having a polar moiety; and
a second polymer;
forming the polymer membrane to comprise at least part of the surfactant molecules and the second polymer, the polymer membrane having a second surface in contact with the first surface; the first polymer, the second polymer and the surfactant molecules being chosen such that the polar moiety has a greater affinity for the first surface than for the second polymer.

2. A method according to claim 1 wherein the step of providing on a first surface of a first polymer comprises:
providing a first composition comprising the surfactant molecules, and a solvent or dispersant to the first surface and evaporating the solvent or dispersant while the first composition resides on the first surface to therewith provide the surfactant molecules to the first surface; and
subsequently, providing a second composition comprising the second polymer, and a solvent or dispersant to the first surface, and evaporating the solvent or dispersant while the second composition resides on the first surface to therewith provide said polymer membrane.
Wherein the solvent or dispersant is not capable of dissolving or dispersing any surfactant molecules already provided to the first surface

3. A method according to claim 1 wherein the step of providing on a first surface of a first polymer comprises:
providing to the first surface a composition comprising: the surfactant molecules, the second polymer and/or precursors for forming the second polymer, and a solvent or dispersant for dissolving and/or dispersing the surfactant molecules, the second polymer and/or the precursors for forming the second polymer; and
evaporating the solvent or dispersant while the composition resides on the first surface to therewith provide said polymer membrane.

4. The method according to claim 1, comprising separating the second surface of the polymer membrane and the first surface of the first polymer.

5. The method according to claim 4, wherein said separating comprises dissolving the first polymer in a solvent in which the polymer membrane is less soluble, or insoluble.

6. The method according to claim 1, wherein the second polymer comprises an elastomer and/or has a Shore A hardness of 70 or less.

7. The method according to claim 1, wherein the second polymer comprises at least one polymer selected from the group consisting of: a polydiene, a polysiloxane, and a polyurethane; optionally wherein the second polymer is chosen from the group consisting of: a polydiene and a polysiloxane.

8. The method according to claim 1 wherein the first polymer comprises a polymer chosen from the group consisting of: a polyol, a polyethyleneglycol and a polyurethane

9. The method of claim 8 wherein the polyol comprises or consists of polyvinylalcohol.

10. The method according to claim 1, wherein the solvent comprises one or more from the group consisting of: a hydrocarbon solvent, an alcohol solvent and a ketone solvent.

11. The method according to claim 1 wherein the polar moieties
are chosen from the group of Bronsted-Lowry acids/bases.

12. The method according to any claim 1 wherein the polar moieties are chosen from the group consisting of: a carboxylic acid group, a phosporic acid group, a phosphorous acid group, and an amine group.

13. The method according to claim 1, wherein the surfactant molecules each comprise a polar moiety chosen from the group consisting of: a carboxylic acid group, an amine group and an amide group.

14. The method according to claim 1, wherein the surfactant molecules are chosen from the group consisting of: a fatty acids, the carboxylic acid groups of the fatty acids defining the polar moiety.

15. The method according to claim 1, further comprising crosslinking the second polymer and at least some of the surfactant molecules.

16. The method according to claim 15, wherein the surfactant molecules comprise one or more reactive groups different form the polar moiety, and the second polymer comprises one or more further reactive groups, the one or more reactive groups and the one or more further reactive groups being chosen to react with each other to therewith crosslink the second polymer and at least some of the surfactant molecules, the method comprising.

17. The method according to claim 15 wherein the second polymer comprises an unsaturated carbon-carbon bond and/or a hydride functional group, and said at least some of the surfactant molecules each comprise an unsaturated carbon-carbon bond, wherein said crosslinking occurs via a reaction between the unsaturated carbon-carbon bond of the surfactant molecules and the unsaturated carbon-carbon bond and/or hydride functional group of the second polymer.

18. The method of claim 1, wherein the second polymer comprises at least one polymer selected from the group consisting of: a polydiene, a polydienestyrene, and a polysiloxane.

19. The method of claim 1 wherein the second polymer is provided such that after formation of the polymer membrane it comprises a thickness less than 100 micrometer, preferably less than 50 micrometer, more preferably less than 20 micrometer.

20. The method according to claim 1, comprising forming one or more apertures and/or grooves in the polymer membrane.

21. The method according to claim 1, comprising mounting one or more polymer membranes in one or more cavities of a body of a cell culturing device.

22. The method according to any of claim 1, comprising functionalizing the second surface with a cell culturing protein.

23. A polymer membrane obtainable from the method according to claim 1.

24. A cell culturing device comprising one or a more of the polymer membrane according to claim 23.

25. A cell culturing device comprising a polymer membrane and a further polymer membrane, the polymer membrane having a surface which opposes a further surface of the further polymer membrane, at least one of the polymer membranes comprising a surfactant comprising surfactant molecules each having a polar moiety at the respective surface or surfaces.

26. A cell culturing method comprising immobilizing cells within a cell culturing device using at least one of said polymer membrane according to claim 24.

27. A method of fabricating a polymer membrane suitable for a cell culture device, the polymer membrane comprising a polymer chosen from the group consisting of a polybutadiene and a polysiloxane, the polymer membrane having a thickness of less than 100 micrometer, the method comprising:
forming one or more apertures and/or grooves in the polymer membrane, using laser ablation with a femto second or picosecond laser.

28. The method of claim 27 wherein the apertures and/or grooves comprise at least one opening dimension measured in the plane of the polymer membrane in a range of less than 50, preferably less than 20 micrometer.

29. The method of claim 27, wherein the wavelength of the femtosecond or picosecond laser is between 400 and 1200 nm.
